(19) United States
(12) Patent Application Publication
(10) Pub. No.: US 2022/0125807 A1
(43) Pub. Date: Apr. 28, 2022

(54) PHARMACEUTICAL COMBINATION OF WNT SIGNALING AND MACC1 INHIBITORS

(71) Applicants: Max-Delbrück-Centrum für Molekulare Medizin in der Holmholtz-Gemeinschafft, Berlin (DE); Charité - Universitätsmedizin Berlin, Berlin (DE)

(72) Inventors: Ulrike Stein, Panketal (DE); Dennis Kobelt, Schildow (DE); Benedikt Kortum, Berlin (DE); Harikrishnan Radhakrishnan, Sunnyvale, CA (US); Wolfgang Walther, Panketal (DE)

(21) Appl. No.: 17/310,756

(22) PCT Filed: Feb. 21, 2020
(86) PCT No.: PCT/EP2020/054641

§ 371 Date: Aug. 20, 2021

(30) Foreign Application Priority Data
Feb. 22, 2019 (EP) 19158805.2

(51) Int. Cl. A61K 31/635; A61K 31/167; A61K 31/192; A61K 31/423; A61K 31/519; A61K 31/63; A61K 31/5415; A61K 31/40; A61K 31/366; A61K 31/405; A61K 31/47; A61K 31/22; A61K 31/505; A61K 31/4184; A61K 31/4523; A61P 35/00

(52) U.S. Cl. CPC A61K 31/635; A61P 35/00; A61K 31/192; A61K 31/423; A61K 31/519; A61K 31/63; A61K 31/5415; A61K 31/40; A61K 31/366; A61K 31/405; A61K 31/47; A61K 31/22; A61K 31/505; A61K 31/4184; A61K 31/4523; A61K 31/167

(57) ABSTRACT

A pharmaceutical combination, includes an inhibitor of the Wnt/β-catenin signaling pathway and an inhibitor of MACC1. One combination includes an inhibitor of S100A4 as a Wnt-signaling inhibitor, preferably niclosamide, and a statin or MEK1 inhibitor as an inhibitor of MACC1. A pharmaceutical composition can include the combination. The combination or composition can be used in the treatment of a tumor disease, such as a solid tumor, and/or for the treatment and/or prophylaxis of tumor metastasis.

The invention relates to a pharmaceutical combination, comprising an inhibitor of the Wnt/β-catenin signaling pathway and an inhibitor of MACC1. In preferred embodiments the invention relates to a combination of an inhibitor of S100A4 as a Wnt-signalling inhibitor, preferably niclosamide, and a statin or MEK1 inhibitor as an inhibitor of MACC1. The invention further relates to a pharmaceutical composition comprising the combination, and use of the combination or composition in the treatment of a tumor disease, such as a solid tumor, and/or for the treatment and/or prophylaxis of tumor metastasis.

### BACKGROUND OF THE INVENTION

Metastatic dissemination of primary tumors is directly linked to patient survival representing the most lethal attribute of cancer. It critically limits successful therapy in many tumor entities. Biomarkers identifying cancer patients at high risk for metastasis and simultaneously acting as key drivers for metastasis are extremely desired. Clinical interventions targeting these molecules are of highest importance.

Until today, about 25-30% of all colorectal cancer (CRC) patients are already distantly metastasized when presenting the first time (stage IV). 40-50% of all patients newly diagnosed with CRC without distant metastases (stages I-III) will develop distant metastasis later (metachronously), after primary surgery, which is significantly linked to shorter survival. 5-year patient survival is about 80% in early stages, but below 10%, when distant metastases occurred. Thus, novel therapeutic options are needed. One important therapeutic goal is the restriction of solid cancer metastasis by novel combinatorial molecular targeted therapies.

The inventors previously identified the novel gene Metastasis-Associated in Colon Cancer (MACC1) in human colorectal cancer (CRC) (Stein et al., Nat Med. 2009 Jan;15(1):59-67). MACC1 induces fundamental processes like proliferation, migration, invasiveness and metastasis in xenografted and transgenic mice. MACC1 has now been established as key factor and as a prognostic and predictive biomarker for tumor progression and metastasis in a variety of solid cancers (CRC, gastric, esophageal, pancreatic, hepatocellular/biliary, lung, nasopharyngeal, renal, bladder, ovarian, breast, cancer, glioblastomas, osteosarcomas). High MACC1 levels in a primary tumor or patient blood predict metastasis formation linked to shorter patient survival.

The inventors previously identified the first MACC1 transcriptional inhibitors (Juneja, Kobelt et al. PLoS Biol. 2017 Jun 1;15(6)). By performing a human MACC1 promoter-based high throughput screen (HTS), FDA-approved small molecule drugs such as lovastatin were identified (FIG. 1) as transcriptional inhibitors of MACC1. These compounds inhibited cell motility in cell culture, and more importantly, restricted metastasis development in xenografted mice. Following this finding, further statins were investigated and identified as inhibitors of MACC1 transcription.

The inventors previously identified the interactome and the phosphointeractome of MACC1. MEK1 was identified as the kinase able and essential to phosphorylate MACC1. MEK inhibitors significantly reduced cell motility in vitro. PoC for MACC1-induced metastasis restriction in mice was demonstrated when deleting the MACC1 pY-sites or inhibiting pY-MACC1 with FDA-approved MEK1 inhibitors such as AZD6244 (selumetinib) and GSK 1120212 (trametinib).

By using isogenic cell line models, the inventors first identified the now established metastasis inducer S100A4 as a transcriptional target of the Wnt/β-catenin signaling pathway (Stein et al., Gastroenterology, 2006 Nov;131(5):1486-500). Thus, intervention targeting the Wnt pathway resulted in reduced S100A4 expression leading to significantly reduced metastasis formation in mice.

The inventors previously identified the first transcriptional inhibitors of the metastasis gene S100A4 using a human S100A4 promoter-based HTS (Stein et al., Neoplasia. 2011 Feb;13(2):131-44; Sack et al. Mol Biol Cell. 2011 Sep;22(18):3344-54; Sack et al., J Natl Cancer Inst. 2011 Jul 6;103(13):1018-36). Among the best candidates, FDA-approved nonsteroidal anti-inflammatory drug sulindac, the antibiotic calcimycin and the anti-helminthic drug niclosamide (WO2012143377) were identified. Interestingly, all of these drugs intervene in the Wnt/β-catenin signaling pathway, reduce cell motility in vitro, and more importantly, significantly restrict metastasis formation in mice.

Translation of these preclinical findings on metastasis inhibition by repositioning of small molecule drugs is currently underway with the FDA-approved anti-helminthic drug niclosamide in a clinical phase II trial: Treatment of stage IV colorectal cancer patients using niclosamide for restriction of tumor progression and metastasis (EudraCT 2014-005151-20, NCT02519582).

The prior art describes the use of niclosamide in order to enhance the bioavailability of peptidic active agents in the treatment atherosclerosis, whereby the administration of pravastatin is also mentioned (WO 2008/021088 A2 and Navab et al, J. Lipid Research, vol. 50, 8, 1538-154, 2009). However, no mention is made of cancer treatment or reduction of cell motility/metastasis.

Despite the substantial roles of MACC1 and the Wnt/β-catenin signaling pathway, in particular S100A4, as prognostic biomarkers and drivers of tumor metastasis, their combinatorial targeting as therapeutic targets for inhibiting tumor and metastasis progression has not been previously investigated in cancer patients.

### SUMMARY OF THE INVENTION

In light of the prior art the technical problem underlying the present invention is to provide alternative or improved means for the treatment of cancer. The technical problem may also be viewed as the provision of means for the treatment and/or reduction of risk of cancer progression in early stage cancer patients. The technical problem may also be viewed as the provision of means for treating and/or reducing the risk of metastasis in patients with solid tumors, more particularly those with elevated MACC1 and/or S100A4 levels.

This problem is solved by the features of the independent claims. Preferred embodiments of the present invention are provided by the dependent claims.

The invention therefore relates to a pharmaceutical combination, comprising
a. an inhibitor of the Wnt/β-catenin signaling pathway, and
b. an inhibitor of MACC1.

The invention also relates to the combination for use in the treatment of a tumor disease, such as a solid tumor, and/or for the treatment and/or prophylaxis of tumor metastasis, and corresponding methods of treatment. The invention also relates to the combined administration of an inhibitor of the Wnt/β-catenin signaling pathway and an inhibitor of MACC1 in such treatment.

The present invention therefore represents a tailored intervention for restriction of cancer progression and metastasis, targeting in combination the recently discovered key driver and prognostic/predictive biomarker MACC1 in combination with the Wnt/β-catenin signaling pathway, a well-known metastasis inducer.

The combined effect of inhibiting both the Wnt/β-catenin signaling pathway and MACC1 leads to an unexpected synergistic effect in reducing cell motility and tumor metastasis.

Additional beneficial effects in reducing tumor cell proliferation are also achieved through the MACC1 inhibitor. The synergy evident in this combined treatment with respect to reduced cell motility and metastasis is quantitatively reproducible. By treating tumor cells with the combination of both an inhibitor of the Wnt/β-catenin signaling pathway and an inhibitor of MACC1, cell motility is reduced to an extent greater than the sum of the effects induced by each inhibitor when administered alone.

Furthermore, this quantitative synergy is evident in both in vitro and in vivo experimental systems. The synergies observed appear therefore to translate into clinical settings, providing effective means in tumor treatment of mammalian, preferably human subjects.

The quantitative synergy is also evident for multiple inhibitors of the Wnt/β-catenin signaling pathway and multiple inhibitors of MACC1. This supports the inventive synergy for the classes of active agents as described herein, without limitation to the particular agents tested. The inventors have established that by interrupting the Wnt/β-catenin signaling pathway beneficial effects in reduced cancer cell motility can be achieved. By combining these effects with an inhibition of MACC1, regardless of the particular pharmaceutical agents employed, that synergistic therapeutic effects, greater than the sum of the individual effects, are achieved. Further detailed descriptions of the quantitative synergistic effects achieved by the combination are evident in the examples, in particular in FIGS. 8-10.

Furthermore, this quantitative synergy is evident at multiple concentrations of various combinations of inhibitors of the Wnt/β-catenin signaling pathway and inhibitors of MACC1. It appears that by varying the relative concentrations of the two agents, no significant loss of quantitative synergy occurs.

In some embodiments, the respective doses of the inhibitor of the Wnt/β-catenin signaling pathway (preferably niclosamide or derivatives thereof) and the inhibitor of MACC1 (preferably a statin) can be reduced compared to usually administered doses. As shown in the examples below, the synergistic effect of the combination of active agents enables lower doses to be administered, for example doses that appear non-efficacious when administered alone show efficacy when administered in the inventive combination. A skilled person could not have derived from common knowledge or the prior art that the inventive combination would allow a more effective and lower dosing of the active agents, thereby potentially maintaining or enhancing efficacy whilst potentially reducing side effects. As is evident from the experimental support provided herein, even low doses of the active agents, for example between 10-50% of the established maximum doses in humans for some active agents, may be employed. Even when administered in such reduced doses, the desired effect of an inhibition of cancer metastasis remains greater than the sum of the effects of the individually dosed components, thereby supporting a synergistic effect.

Additionally, MACC1 induces S100A4 expression and secretion (FIGS. 2-4). The inventors have therefore identified a novel link between these two molecular targets. As such, the observed synergy obtained via the combination of the present invention represents an unexpected synergy. A functional interaction between MACC1 and S100A4 has not been previously described with respect to cancer proliferation or metastasis.

Furthermore, a skilled person would not have expected these two molecular targets to interact beyond a summative effect. Even if a connection between MACC1 and the Wnt/β-catenin signaling pathway could have been derived from the art, which is to the knowledge of the inventors not evident, a synergistic effect by targeting these factors could not have been predicted based on the expectation of a skilled person. Typically, targeting two factors in related pathways would not typically lead to synergistic effects, but to no additional or negligible additive effects. Considering MACC1 and S100A4 are mechanistically related, a skilled person would expect the opposite as has been observed in the present invention. Without being bound by theory, a second (combined) factor would typically lead to no additional or at most a small summative effect, as targeting two positions in the same pathway or related pathways would typically not lead to further quantitative effects, as the pathway has already been inhibited by the first agent, such that by targeting another position in the same or a related pathway by a second agent would not have an additional effect, but merely reinforce the effect already achieved by the first agent. Contrary to expectations, a surprising and quantitative synergy has been achieved by inhibiting both MACC1 and the Wnt/β-catenin signaling pathway.

In one embodiment, the invention relates to a pharmaceutical combination, comprising a. an inhibitor of the Wnt/β-catenin signaling pathway, and b. an inhibitor of MACC1, wherein the inhibitors a. and b. achieve a synergistic effect in reducing cell motility and/or tumor metastasis.

In one embodiment, the inhibitors a. and b. achieve a synergistic effect in reducing cell motility in an in vitro wound healing assay, preferably as described in the examples below.

In one embodiment, the inhibitors a. and b. achieve a synergistic effect in reducing cell motility in an in vitro wound healing assay using HCT116 cells.

In one embodiment, the synergy is determined using the Loewe or the HSA model. In other embodiments, synergy is determined using the Bliss model and/or the ZIP model.

In one embodiment, the inhibitor of the Wnt/β-catenin signaling pathway is an inhibitor of S100A4.

The Wnt/β-catenin signaling pathway target gene S100A4 is overexpressed in many different types of cancer, such as gallbladder, bladder, breast, esophageal, gastric, pancreatic, hepatocellular, non-small cell lung and especially colorectal cancer. Increased expression of S100A4 is strongly associated with aggressiveness of a tumor, its ability to metastasize and poor survival in patients. Inhibition of S100A4 has also been shown to lead to inhibit S100A4-induced cell migration and invasion as well as cell proliferation and colony formation in vitro.

In one embodiment, the inhibitor of the Wnt/β-catenin signaling pathway is niclosamide or a derivative thereof, sulindac, calcimycin, ICG001, FH535, LF3, a phenothiazine, such as trifluoperazine (TFP), or functionally analogous derivative thereof.

Niclosamide, sold under the trade name Yomesan among others, is a medication used originally to treat tapeworm infestations. Niclosamide is also known as 5-Chloro-N-(2-chloro-4-nitrophenyl)-2-hydroxybenzamide.

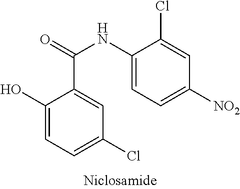 [SMILES: O=C(Nc1ccc([N+](=O)[O-])cc1Cl)c1cc(Cl)ccc1O]

Niclosamide has also been shown to exhibit strong inhibitory effects in suppressing motility and metastasis of cancerous cells expressing S100A4. Niclosamide effectively inhibits the expression of S100A4, wherein it modulates the TCF/beta-catenin protein complex. The S100A4 gene is a target of the Wnt pathway and its expression is activated by the transcription complex TCF/beta-catenin. Modulation of the transcription complex TCF/beta-catenin via niclosamide treatment results in a stop or inhibition of the transcription of the S400A4 gene. The effect of niclosamide inhibiting S100A4 was discovered by a high-throughput screen of small molecules to identify inhibitors of S100A4 promoter-driven reporter gene expression with potential clinical anti-metastatic activity.

Various derivatives of niclosamide have been shown to be effective on S100A4-driven cell migration, invasion and metastasis, and are encompassed in the present application. The person skilled in the art knows various chemical methods and techniques to render a chemical substance to generate a derivate, which still comprises the chemical basis, such as addition, deletion or substitution of a group or functional group. Derivatives include those described in e.g. WO2012143377.

Examples of niclosamide derivatives are, without limitation:

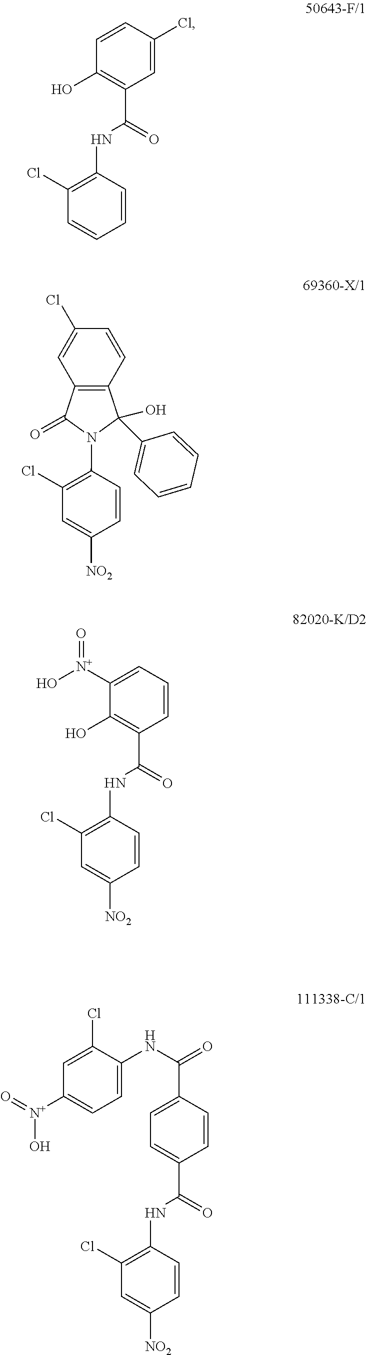

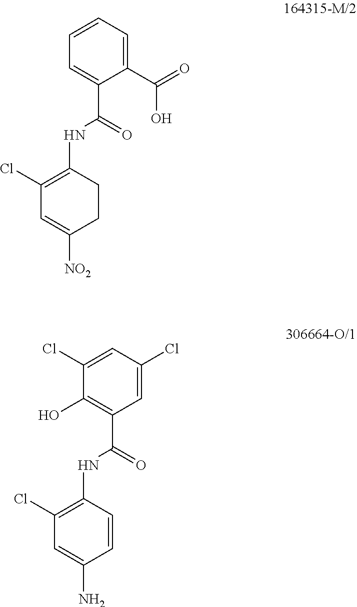 [SMILES: O=C(O)c1ccccc1C(=O)NC1=C(Cl)C=C([N+](=O)[O-])CC1 ; Nc1ccc(NC(=O)c2cc(Cl)cc(Cl)c2O)c(Cl)c1]

Further examples of niclosamide derivatives are, without limitation:

DK419, as disclosed in Wang et al (Bioorg Med Chem. 2018 Nov 1;26(20):5435-5442):

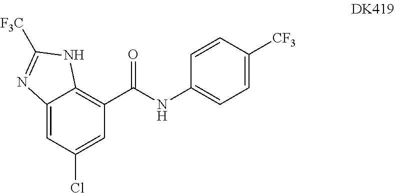 [SMILES: O=C(Nc1ccc(C(F)(F)F)cc1)c1cc(Cl)cc2nc(C(F)(F)F)[nH]c12]

A17 or B17, as disclosed in Wu et al (Cancer Med. 2018 Aug;7(8):3945-3954):

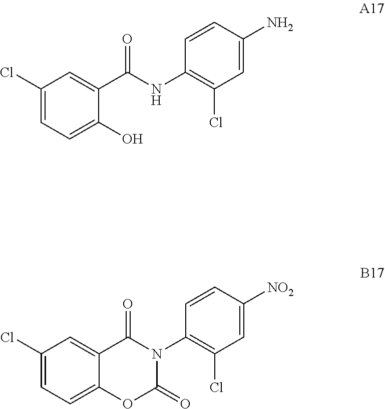 [SMILES: Nc1ccc(NC(=O)c2cc(Cl)ccc2O)c(Cl)c1 ; O=c1oc2ccc(Cl)cc2c(=O)n1-c1ccc([N+](=O)[O-])cc1Cl]

Analog 11 or 32, as disclosed in Arend et al (Oncotarget. 2016 Dec 27;7(52):86803-86815):

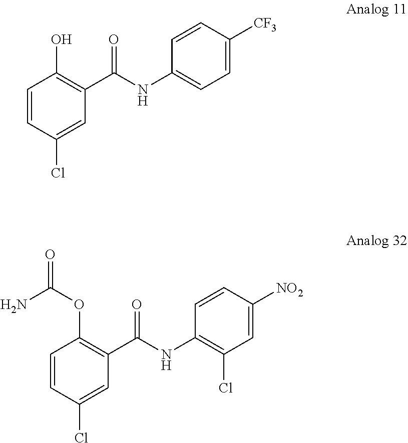 [SMILES: O=C(Nc1ccc(C(F)(F)F)cc1)c1cc(Cl)ccc1O ; NC(=O)Oc1ccc(Cl)cc1C(=O)Nc1ccc([N+](=O)[O-])cc1Cl]

Sulindac is a nonsteroidal anti-inflammatory drug (NSAID) of the arylalkanoic acid class that is marketed in the UK & U.S. by Merck as Clinoril. Like other NSAIDs, it is useful in the treatment of acute or chronic inflammatory conditions. Sulindac was identified as a transcriptional inhibitor of the metastasis gene S100A4 using a human S100A4 promoter-based high throughput screen.

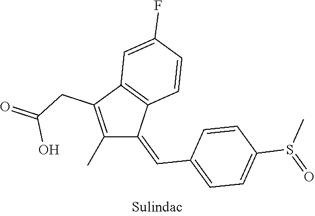 [SMILES: CC1=C(CC(=O)O)c2cc(F)ccc2/C1=C\c1ccc(S(C)=O)cc1]

Calcimycin is an ionophorous, polyether antibiotic from Streptomyces chartreusensis. It binds and transports calcium and other divalent cations across membranes and uncouples oxidative phosphorylation while inhibiting ATPase of rat liver mitochondria. Calcimycin is also known as A23187, Calcium lonophore, Antibiotic A23187 and Calcium lonophore A23187. It is produced at fermentation of Streptomyces chartreusensis. The inventors have demonstrated that Calcimycin also inhibits S100A4.

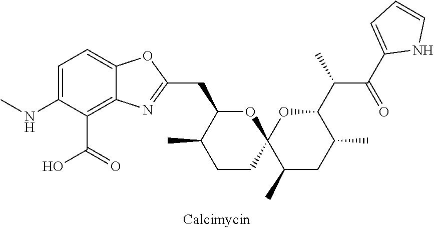 [SMILES: CNc1ccc2oc(C[C@H]3O[C@@]4(CC[C@H]3C)O[C@H]([C@H](C)C(=O)c3ccc[nH]3)[C@H](C)C[C@H]4C)nc2c1C(=O)O]

Additional inhibitors of S100A4 are known in the art. For example, 6-B12 is under investigation for the treatment of metastatic cancer and mammary tumor with metastasis to the lung. 6-B12 is a monoclonal antibody that acts against S100A4 (Cancer Research UK). 6-B12 has been shown to neutralize S100A4, suppress spontaneous tumor progression and pre-metastatic niche formation, and alter T-cell polarization balance. In a mouse model of spontaneous breast cancer effects on the dynamics of tumor growth and metastasis were assessed. In a model of metastatic niche formation, the expression of metastatic niche markers was determined. The S100A4 blocking antibody 6-B12 reduces tumor growth and metastasis in a model of spontaneous breast cancer.

The 6612 antibody inhibits T cell accumulation at the primary and pre-metastatic tumor sites. The 6612 antibody further acts as an immunomodulatory agent (Grum-Schwensen et al, BMC Cancer. 2015; 15: 44).

LK-1 is under development for the treatment of solid tumors and pancreatic cancer. LK-1 is a humanized monoclonal antibody that acts by targeting S100A4 (Lykera Biomed).

ICG001 antagonizes Wnt/β-catenin/TCF-mediated transcription and specifically binds to CREB-binding protein (CBP). By binding CBP, thus blocking interaction with β-catenin, it selectively induces apoptosis in transformed colon cells but not in normal cells and prevents the growth of colon carcinoma cells.

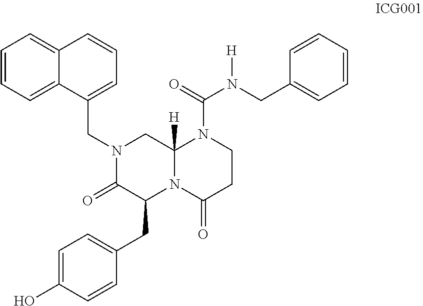 [SMILES: O=C1[C@H](Cc2ccc(O)cc2)N2C(=O)CCN(C(=O)NCc3ccccc3)[C@H]2CN1Cc1cccc2ccccc12]

FH535 is a reversible dual inhibitor of Wnt/β-catenin and a compound that suppresses both Wnt/beta-catenin and peroxisome proliferator-activated receptor (PPAR) signaling. FH535 is unique in its ability to inhibit the Wnt/β-catenin pathway. The compound is selectively toxic to some carcinomas expressing the Wnt/β-catenin pathway.

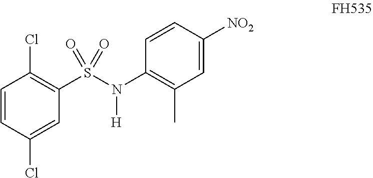 [SMILES: Cc1cc([N+](=O)[O-])ccc1NS(=O)(=O)c1cc(Cl)ccc1Cl]

LF3 is a specific inhibitor of canonical Wnt signaling by disrupting the interaction between β-catenin and TCF4. LF3 does not cause cell death or interfere with cadherin-mediated cell-cell adhesion. The self-renewal capacity of cancer stem cells is blocked by LF3 in concentration-dependent manners. LF3 blocks the expression of a series of Wnt target genes in Wnt-addicted colon cancer cells. It inhibits proliferation of Wnt-addicted colon cancer cells through induction of cell-cycle arrest and also inhibits self-renewal capacity of CSCs.

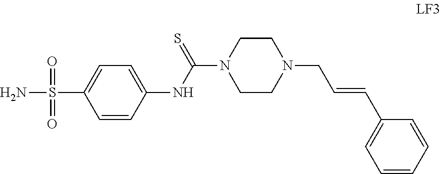 [SMILES: NS(=O)(=O)c1ccc(NC(=S)N2CCN(C/C=C/c3ccccc3)CC2)cc1]

Phenothiazines inhibit S100A4 function by inducing protein oligomerization. Phenothiazines, such as trifluoperazine (TFP), have been shown to inhibit S100A4 (Malashkevich et al, PNAS, 2010, vol. 107, no. 19, 8605-8610). Trifluoperazine (TFP) was identified as an inhibitor that disrupts the S100A4/myosin-IIA interaction.

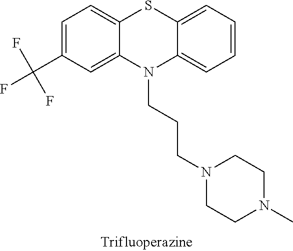 [SMILES: CN1CCN(CCCN2c3ccccc3Sc3ccc(C(F)(F)F)cc32)CC1]

In one embodiment, the inhibitor of MACC1 is a statin.

Statins, also known as HMG-CoA reductase inhibitors, are a class of lipid-lowering medications. They reduce cardiovascular disease and mortality in those who are at high risk of cardiovascular disease. Statins are effective in lowering LDL cholesterol and statins are therefore widely used for primary prevention in people at high risk of cardiovascular disease, as well as in secondary prevention for those who have developed cardiovascular disease. The inventors have now shown that statins lead to an inhibition of MACC1. As is shown in more detail in the examples below, all tested statins were able to reduce MACC1 mRNA expression and MACC1 protein. The inventors confirm that the all statins tested are able to reduce cellular motility when applied as monotherapy in the wound healing assay. A dose dependent reduction of wound closure over time was observed (FIG. 7).

In one embodiment, the inhibitor of MACC1 is a statin selected from atorvastatin, lovastatin, fluvastatin, pitarvastatin, pravastatin, rosuvastatin and/or simvastatin, preferably atorvastatin, lovastatin and/or fluvastatin.

It was entirely surprising that statins would exhibit an effect as described herein, namely that statins would reduce MACC1 mRNA expression and MACC1 protein, and additionally that statins are able to reduce cellular motility when applied as monotherapy in a wound healing assay. The combinatorial synergy with Wnt-signaling inhibitors (such as niclosamide) also represents an unexpected finding, as a combined beneficial effect could not be derived from any suggestion in the art.

Atorvastatin, sold under the trade name Lipitor among others, is a statin medication used to prevent cardiovascular disease in those at high risk and treat abnormal lipid levels. Atorvastatin is shown to reduce MACC1 mRNA expression and MACC1 protein levels.

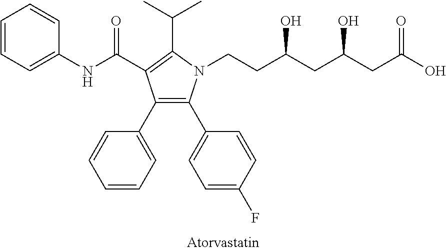 [SMILES: CC(C)c1c(C(=O)Nc2ccccc2)c(-c2ccccc2)c(-c2ccc(F)cc2)n1CC[C@@H](O)C[C@@H](O)CC(=O)O]

Lovastatin (Mevacor) is a statin drug, used for lowering cholesterol in those with hypercholesterolemia to reduce risk of cardiovascular disease. Lovastatin is shown to reduce MACC1 mRNA expression and MACC1 protein levels.

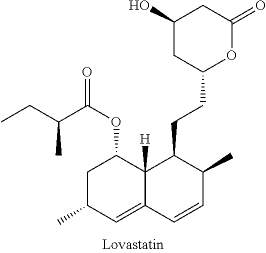 [SMILES: CC[C@H](C)C(=O)O[C@H]1C[C@@H](C)C=C2C=C[C@H](C)[C@H](CC[C@@H]3C[C@@H](O)CC(=O)O3)[C@H]21]

Fluvastatin (Lescol, Canef, Vastin) is a member of the statin drug class, used to treat hypercholesterolemia and to prevent cardiovascular disease. Fluvastatin is shown to reduce MACC1 mRNA expression and MACC1 protein levels.

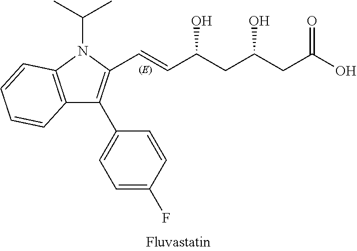 [SMILES: CC(C)n1c(/C=C/[C@H](O)C[C@H](O)CC(=O)O)c(-c2ccc(F)cc2)c2ccccc21]

Pitavastatin is a member of the blood cholesterol lowering medication class of statins, marketed in the United States under the trade name Livalo. Like other statins, it is an inhibitor of HMG-CoA reductase, the enzyme that catalyses the first step of cholesterol synthesis. Pitavastatin is shown to reduce MACC1 mRNA expression and MACC1 protein levels.

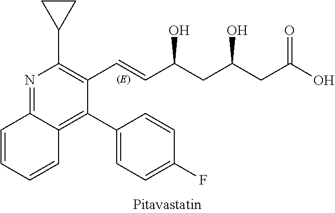 [SMILES: O=C(O)C[C@H](O)C[C@H](O)/C=C/c1c(C2CC2)nc2ccccc2c1-c1ccc(F)cc1]

Pravastatin (Pravachol) is a statin medication, used preventing cardiovascular disease in those at high risk and treating abnormal lipids. Pravastatin is capable of reducing MACC1 mRNA expression and/or MACC1 protein levels.

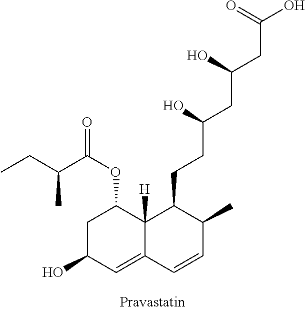 [SMILES: CC[C@H](C)C(=O)O[C@H]1C[C@H](O)C=C2C=C[C@H](C)[C@H](CC[C@@H](O)C[C@@H](O)CC(=O)O)[C@H]21]

Rosuvastatin (Crestor) is a statin medication, used to prevent cardiovascular disease in those at high risk and treat abnormal lipids. Rosuvastatin is capable of reducing MACC1 mRNA expression and/or MACC1 protein levels.

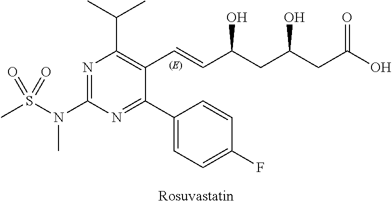 [SMILES: CC(C)c1nc(N(C)S(C)(=O)=O)nc(-c2ccc(F)cc2)c1/C=C/[C@@H](O)C[C@@H](O)CC(=O)O]

Simvastatin (Zocor) is a lipid-lowering medication. It is used along with exercise, diet, and weight loss to decrease elevated lipid (fat) levels. Simvastatin is capable of reducing MACC1 mRNA expression and/or MACC1 protein levels.

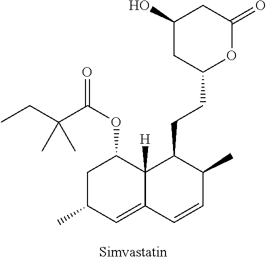 [SMILES: CCC(C)(C)C(=O)O[C@H]1C[C@@H](C)C=C2C=C[C@H](C)[C@H](CC[C@@H]3C[C@@H](O)CC(=O)O3)[C@H]21]

The above statins are provided as non-limiting preferred embodiments of the statin class of compounds. Until now, the multiple statins tested by the inventors have achieved the desired MACC1 inhibition in addition to the desired synergy in reducing cell motility when used in combination with an inhibitor of the Wnt/β-catenin signaling pathway, preferably niclosamide.

In one embodiment, the inhibitor of MACC1 is a MEK1 inhibitor.

As shown below, the MEK inhibitors tested herein, preferably the MEK1 inhibitors AZD6244 (selumetinib), GSK1120212 (trametinib) or cobimetinib, lead to the desired MACC1 inhibition and synergy with an inhibitor of the Wnt/β-catenin signaling pathway, preferably niclosamide. MACC1 is phosphorylated by MEK1, leading to induction of the MACC1 mediated effects. Therefore, MEK1 inhibitors, such as GSK1120212 (trametinib, approved for melanoma treatment) and AZD6244 (selumetinib) act to reduce MACC1 activity, and also act synergistically on cellular motility when combined with an inhibitor of the Wnt/β-catenin signaling pathway, preferably niclosamide.

A MEK inhibitor is a chemical or drug that inhibits the mitogen-activated protein kinase enzymes MEK1 and/or MEK2. Inhibitors of MEK1 and MEK2 are comprised in the invention. They can be used to affect the MAPK/ERK pathway, which is overactive in some cancers.

As non-limiting examples of MEK inhibitors, selumetinib (AZD6244) is a drug being investigated for the treatment of various types of cancer, such as non-small cell lung cancer (NSCLC) and thyroid cancer. MACC1 is phosphorylated by MEK1 and selumetinib inhibits MEK1, leading to a reduction in MACC1 mediated effects and a reduction in cell proliferation and/or motility.

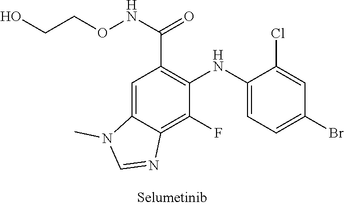 [SMILES: Cn1cnc2c(F)c(Nc3ccc(Br)cc3Cl)c(C(=O)NOCCO)cc21]

Trametinib (GSK1120212), is FDA-approved to treat BRAF-mutated melanoma. Trametinib (Mekinist) is a MEK inhibitor drug with anti-cancer activity and inhibits MEK1 and MEK2. MACC1 is phosphorylated by MEK1 and trametinib inhibits MEK1, leading to a reduction in MACC1 mediated effects and a reduction in cell proliferation and/or motility.

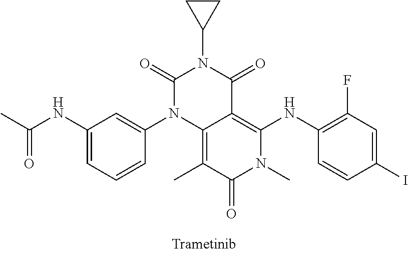 [SMILES: CC(=O)Nc1cccc(-n2c(=O)n(C3CC3)c(=O)c3c(Nc4ccc(I)cc4F)n(C)c(=O)c(C)c32)c1]

Cobimetinib or XL518, is approved by US FDA for use in combination with vemurafenib (Zelboraf(R)), for treatment of advanced melanoma with a BRAF V600E or V600K mutation. MACC1 is phosphorylated by MEK1 and cobimetinib inhibits MEK1, leading to a reduction in MACC1 mediated effects and a reduction in cell proliferation and/or motility.

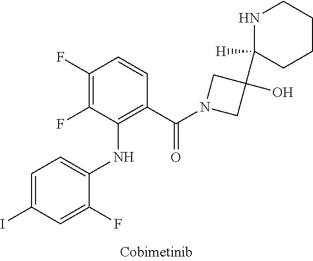 [SMILES: O=C(c1ccc(F)c(F)c1Nc1ccc(I)cc1F)N1CC(O)([C@@H]2CCCCN2)C1]

Binimetinib (MEK162), was approved by the FDA in 2018 in combination with encorafenib for the treatment of patients with unresectable or metastatic BRAF V600E or V600K mutation-positive melanoma.

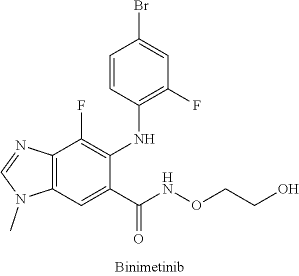 [SMILES: Cn1cnc2c(F)c(Nc3ccc(Br)cc3F)c(C(=O)NOCCO)cc21]

In some embodiments, the combination described herein comprises two or more (or at least two) separate compounds. In some embodiments, the inhibitor of the Wnt/β-catenin signaling pathway may also be an inhibitor of MACC1 (in some embodiments to some residual extent). In some embodiments, the inhibitor of MACC1 may also be an inhibitor of the Wnt/β-catenin signaling pathway (in some embodiments to some residual extent).

Preferred MACC1 inhibitors have an inhibitory effect on MACC1 protein function (post-translational inhibitors), preferably by inhibiting MACC1 phosphorylation (in some embodiments by inhibiting a mitogen-activated protein kinase enzyme (MEK). Preferred Wnt-signaling inhibitors have an inhibitory effect on S100A4 transcription, preferably by disrupting the transcription complex TCF/beta-catenin (e.g. via niclosamide or derivatives thereof). In some embodiments, the inhibitor of MACC1 has a smaller inhibitory effect on S100A4 transcription than niclosamide, i.e. the MACC1 inhibitor acts primarily on MACC1 protein function with a smaller effect on S100A4 transcription than the preferred Wnt-signalling inhibitors described herein.

In one embodiment, the invention relates to a pharmaceutical combination as described herein, comprising:
a. niclosamide or derivative thereof, preferably as disclosed herein, and
b. a statin and/or a MEK1 inhibitor.

The combinations of niclosamide with either a statin and/or a MEK1 inhibitor represent preferred embodiments, for which synergistic effects have been demonstrated in the examples below. It was entirely surprising that the quantitative synergies in reducing motility and metastasis of cancerous cells could be achieved through these combinations.

In preferred embodiments, the combination is selected from the group consisting of niclosamide and atorvastatin, niclosamide and lovastatin, niclosamide and fluvastatin, niclosamide and AZD6244 (selumetinib) or niclosamide and GSK1120212 (trametinib).

In some embodiments, the inhibitor of the Wnt/β-catenin signaling pathway (a.) and the inhibitor of MACC1 (b.) have relative amounts of 10000:1 to 1:10000 by weight, preferably 5000:1 to 1:1 by weight.

As demonstrated in the examples below, the relative concentrations of the combined agents tested show that no loss of synergy occurs when testing the agents at various relative concentrations. As such, the invention encompasses any relative concentration and/or amount of the two classes of agents disclosed herein.

In one embodiment, (a.) the inhibitor of the Wnt/β-catenin signaling pathway is niclosamide and (b.) the inhibitor of MACC1 is a statin, preferably atorvastatin, lovastatin, fluvastatin, pitarvastatin, pravastatin, rosuvastatin and/or simvastatin, and (a.) and (b.) have relative amounts of 1000:1 to 1:1, preferably 500:1 to 1:1, more preferably 50:1 to 10:1, more preferably about 25:1.

In one embodiment, (a.) the inhibitor of the Wnt/β-catenin signaling pathway is niclosamide and (b.) the inhibitor of MACC1 is a MEK1 inhibitor, and (a.) and (b.) have relative amounts of 5000:1 to 1:1, more preferably 2000:1 to 500:1.

In one embodiment, (a.) the inhibitor of the Wnt/β-catenin signaling pathway is niclosamide and (b.) the MEK1 inhibitor is GSK1120212 (trametinib), and (a.) and (b.) have relative amounts of 2000:1 to 500:1, preferably about 1000:1,

In one embodiment, (a.) the inhibitor of the Wnt/β-catenin signaling pathway is niclosamide and (b.) the MEK1 inhibitor is AZD6244 (selumetinib) or cobimetinib, and (a.) and (b.) have relative amounts of 1000:1 to 1:1, preferably 500:1 to 1:1, more preferably 50:1 to 10:1, more preferably about 35:1 to 25:1.

The above embodiments regarding particular relative amounts are based on clinically allowed or currently trialed doses of the various compounds described herein, being combined into a pharmaceutical combination as described herein.

In preferred embodiments, the agents a. and b. (the inhibitor of the Wnt/β-catenin signaling pathway (a.) and the inhibitor of MACC1 (b.)) are administered in concentrations or amounts sufficient to provide a therapeutic effect. A skilled person can determine such an effect without undue effort. This amount relates to a therapeutically effective amount when an agent is used alone, or to a therapeutically effective amount when an agent is used in combination with a second agent. In preferred embodiments, the agents are administered in concentrations or amounts, or according to dosage regimes, already established in the art, such as those for which regulatory approval has been issued (e.g. by the FDA or EMA), or in doses currently being assessed during phase 2 or 3 clinical trials, and/or according to the maximum allowed dose according to a phase I trial.

In such a treatment, agent (a.) and agent (b.) of the combination may be administered simultaneously or sequentially to said patient, agent (a.) being indifferently administered before or after agent (b.). Agent (a.) and agent (b.) may also be administered by the same or a different administration route.

In one embodiment, niclosamide is administered to a human subject in an amount of 100 mg to 5000 mg, preferably 500 mg to 3000 mg, more preferably 500 mg to 2000 mg, or 1000 to 2000 mg, or 1500 mg to 2500 mg, preferably about 1000, 1500 or 2000 mg per day. In preferred embodiments, the dose of niclosamide is an oral, daily dose of between 1000-3000 mg, preferably between 1500-2500, more preferably 2000 mg.

In one embodiment, one or more statin(s) is (are) administered to a human subject in an amount of 5 mg to 500 mg, preferably 20 mg to 200 mg, more preferably 50 mg to 150 mg, more preferably about 80 mg per day. These embodiments relate individually to one or more of atorvastatin, lovastatin, fluvastatin, pitarvastatin, pravastatin, rosuvastatin and/or simvastatin.

In one embodiment, one or more statin(s), preferably atorvastatin, lovastatin and/or fluvastatin, is (are) administered to a human subject in an amount of 1 mg to 500 mg, or 5 mg to 500 mg, preferably 20 mg to 200 mg, more preferably 50 mg to 150 mg, more preferably about 80 mg, orally in a daily dose, in combination with niclosamide, in an oral, daily dose of between 500-3000 mg, preferably between 800-2500, more preferably 1000-2000 mg.

In one embodiment, one or more MEK inhibitors (MEK1 or MEK2 inhibitors) is(are) administered to a human subject in an amount of 0.1 mg to 500 mg, preferably 1 mg to 200 mg, more preferably 20 mg to 150 mg, more preferably about 2, 50, 55, 60, 65, 70, 75, 80, 85, or 90 mg per day.

In preferred embodiments, trametinib is administered to a human subject in an amount of 0.1 mg to 20 mg, preferably 1 mg to 10 mg, more preferably 1 mg to 5 mg, more preferably about 2 mg per day.

In preferred embodiments, selumetinib is administered to a human subject in an amount of 1 mg to 500 mg, preferably 10 mg to 200 mg, more preferably 20 mg to 150 mg, more preferably about 75 mg per day.

In preferred embodiments, cobimetinib is administered to a human subject in an amount of 1 mg to 500 mg, preferably 10 mg to 200 mg, more preferably 20 mg to 150 mg, more preferably about 60 mg per day.

A further aspect of the invention relates to the pharmaceutical combination as described herein for use in the treatment of a tumor disease.

The invention therefore relates to a method for treating a tumor disease in a subject in need thereof, or a patient at risk of suffering from a tumor disease. The method of treatment preferably comprising administering to said subject a therapeutically effective amount of the pharmaceutical combination, or the two agents of the combination, in order to obtain a therapeutic effect.

A further aspect of the invention therefore relates to an inhibitor of the Wnt/β-catenin signaling pathway, preferably niclosamide, sulindac, calcimycin, ICG001, FH535, LF3 and/or a phenothiazine, such as trifluoperazine (TFP), more preferably niclosamide, for use as a medicament in the treatment of a tumor disease, preferably for the treatment of tumor metastasis, a solid tumor or a particular tumor disease disclosed herein, wherein said treatment comprises the combined administration of an inhibitor of MACC1, preferably a statin or a MEK1 inhibitor, more preferably according to atorvastatin, lovastatin, fluvastatin, pitarvastatin, pravastatin, rosuvastatin and/or simvastatin.

A further aspect of the invention therefore relates to an inhibitor of MACC1, preferably a statin or a MEK1 inhibitor, more preferably atorvastatin, lovastatin, fluvastatin, pitarvastatin, pravastatin, rosuvastatin and/or simvastatin, or more preferably AZD6244 (selumetinib), GSK1120212 (trametinib) or cobimetinib, for use as a medicament in the treatment of a tumor disease, preferably for the treatment of tumor metastasis, a solid tumor or specific tumor disease described herein, wherein said treatment comprises the combined administration of an inhibitor of the Wnt/β-catenin signaling pathway, preferably niclosamide, sulindac, calcimycin, ICG001, FH535, LF3 or a phenothiazine, such as trifluoperazine (TFP), more preferably niclosamide.

In one embodiment, the invention relates to the pharmaceutical combination, or the two agents of the combination, for use in the treatment and/or prophylaxis of tumor metastasis, preferably by reducing cellular motility of cancerous cells.

As is shown in the examples below, the synergistic effect is evidenced via both in vitro and in vivo models. The in vitro approach employed uses a wound healing assay with the HCT116 human colon cancer cell line. HCT116 cells are endogenously positive for MACC1 and S100A4 expression at the mRNA and protein level. The synergistic effect is quantified based on the direct effect on reduced cell motility of HCT116 cells observed in the wound healing assay. Cell motility is a fundamental and ancient cellular behavior that contributes directly to the pathology of metastasis. By exhibiting an effect on cell motility, as evidenced by the wound healing assay, the therapeutic setting of treating and/or preventing metastasis is supported. Synergistic effects may also be observed for reducing cancer cell migration and/or invasion.

In one embodiment, the tumor is a solid tumor, preferably gastrointestinal, colorectal, gastric, esophageal, pancreatic, hepatocellular, biliary, lung, nasopharyngeal, renal, bladder, ovarian, brain, bone or breast cancer.

Solid tumors have been analyzed previously by the inventors and others and characterized for their responsiveness to inhibitors of the Wnt/β-catenin signaling pathway and/or inhibitors of MACC1. The solid tumors listed above are known to respond to e.g. niclosamide. The solid tumors listed above are known to exhibit expression of MACC1 and/or show active Wnt/β-catenin signaling, preferably evident via S100A4 expression.

In one embodiment, the tumor cells exhibit increased expression and/or activity of MACC1 and S100A4 compared to a control, such as healthy controls.

The inventors demonstrate herein, in tumors as well as in patient's blood, the beneficial role of combining both biomarkers for improved prognosis for CRC (Stein et al. 2012, PLoS One. 2012;7(11)) and gastric cancer patients (Burock et al. World J Gastroenterol 2015 January 7; 21(1): 333-341). A significant benefit for patients exhibiting increased MACC1 and S100A4 expression and/or activity is evident when treated with the pharmaceutical combination as described herein, i.e. a drug combination acting on both molecules (FIG. 4 shows additional survival rates of patients based on MACC1 and S100A4 expression levels).

In some embodiments, drug-induced S100A4 and/or MACC1 modulation and therapy efficacy can also be monitored for therapy response using blood-based S100A4 and/or MACC1 assays in conjunction with HPLC determination of niclosamide in patient blood.

In a preferred embodiment the pharmaceutical combination for use as a medicament as described herein is characterized in that the treatment of cancer comprises the treatment of a subject with elevated levels of MACC1 and S100A4 compared to a control, such as healthy controls.

A skilled person is capable of determining elevated levels of MACC1 and S100A4 using standard techniques. By way of example, a method may be employed selected from the group consisting of nucleic acid amplification methods, such as PCR, qPCR, RT-PCR, qRT-PCR or isothermal amplification, mass spectrometry (MS), luminescence immunoassay (LIA), radioimmunoassay (RIA), chemiluminescence- and fluorescence- immunoassays, enzyme immunoassay (EIA), Enzyme-linked immunoassays (ELISA), luminescence-based bead arrays, magnetic beads based arrays, protein microarray assays, rapid test formats such as for instance immunochromatographic strip tests, rare cryptate assay, and automated systems/analyzers.

Elevated levels of MACC1 and S100A4 may be determined in comparison to an appropriate control. Control samples or patients may relate to a healthy control subject or samples therefrom, such as a subject with no detectable cancer or history of cancer. When considering the risk of metastasis, a control may also involve the use of samples from patients with cancer (for example those specific cancers described herein), but with no metastasis. As a positive control, samples from patients with elevated MACC1 and S100A4 expression and/or extensive metastasis may be used.

Elevated levels of MACC1 and/or S100A4 may be determined via nucleic acid and/or protein levels. Increased amounts of MACC1 and/or S100A4 protein and/or mRNA transcripts may be used to detect the levels of MACC1 and/or S100A4 in patients or control subjects. MACC1 and S100A4 are expressed in tumour tissue. Biopsy samples or removed tumors may be used to measure MACC1 and/or S100A4 levels as described herein. Furthermore, MACC1 and/or S100A4 protein, and/or MACC1 and/or S100A4 encoding nucleic acids, are detectable in bodily fluids of patients, such as urine or blood, or samples derived from blood such as blood plasma or serum, of patients. Specific methods, useful in the invention but not limiting to the invention, for identifying elevated levels of MACC1 and/or S100A4 are described in more detail below.

The treatment of patients with elevated levels of MACC1 and/or S100A4 may, in some embodiments, comprise a combined method incorporating diagnosis of a cancer on the basis of elevated MACC1 and/or S100A4 levels, for example an elevation in MACC1 and/or S100A4 transcript levels, and subsequent treatment with the combination described herein.

The treatment of patients with elevated levels of MACC1 and/or S100A4 represents a novel clinical situation that arises from the MACC1 and Wnt-signaling inhibition described herein.

Although MACC1 and S100A4 were known to be prognostic markers for cancer metastasis, treatment based on the provision of MACC1 and Wnt-signaling (S100A4) inhibitors has not previously been proposed.

Patients who have been diagnosed with cancer or with increased risk of developing cancer or metastases on the basis of MACC1 and/or S100A4 levels can now be treated directly via interrupting MACC1 and S100A4 function. The invention therefore enables efficient therapeutic approaches for those patients with elevated MACC1 and/or S100A4 levels. The inhibitors of the present invention enable effective treatment according to a novel patient stratification strategy. Those patients without elevated MACC1 and/or S100A4 levels are typically not selected for treatment with the compounds disclosed herein, thereby making administration of the combination described herein targeted towards a population of patients with elevated MACC1 and/or S100A4 levels who will likely respond to the treatment, thereby saving time and financial cost of treating non-responders without elevated MACC1 and/or S100A4 levels.

The group of patients defined by elevated MACC1 and/or S100A4 levels compared to a suitable control group represents a patient group that is defined by a clear pathological and physiological criterion that is directly related to disease occurrence and progression. Furthermore, no indication has been provided in the art that this patient group could be treated by an inhibitor combination directed specifically to the disease-causing and disease-indicative molecules MACC1 and S100A4 themselves. It was entirely unexpected that an effective treatment could be developed for this patient group, based on synergistic effects of combined MACC1 and S100A4 targeting.

In a preferred embodiment the MACC1-Inhibitor as described herein is a post-translational MACC1 inhibitor. For example, the MEK inhibitors lead to inhibition of MACC1 phosphorylation, thereby leading to reduced activity. The reduction in activity may be determined by comparison to an appropriate control, such as other subjects with elevated MACC1 activity and/or subjects not undergoing said treatment. In other embodiments, transcriptional repressors of MACC1, such as Rottlerin, may be employed.

In a preferred embodiment the inhibitor of the Wnt/β-catenin signaling pathway is a transcriptional repressor of S100A4, causing preferably inhibition or repression of transcription of a S100A4-encoding nucleic acid. The reduction in transcription may be determined by comparison to an appropriate control, such as other subjects with elevated S100A4levels and/or subjects not undergoing said treatment.

The MACC1-Inhibitors and the inhibitors of the Wnt/β-catenin signaling pathway may be characterized as anti-metastatic agents. The anti-metastatic effect can be characterized in that the cancer treatment of the present invention comprises preventing and/or reducing cancer cell metastasis and/or cell migration or motility in a subject receiving treatment.

In particular, the preventative effect on reducing the risk of metastasis is a unique feature of the present invention. Patients previously diagnosed with elevated levels of MACC1 and/or S100A4 were associated with higher risks of metastasis, without having any appropriate “prophylactic” therapy. The identification of MACC1 and S100A4 inhibition by the combination described herein now enables these patients to be effectively and directly treated in advance of cancer metastasis.

In some embodiments, the MACC1-Inhibitor for use as a medicament as described herein may be characterized as an anti-proliferative agent. The anti-proliferative effect can be characterized in that the cancer treatment of the present invention comprises preventing and/or reducing cancer cell proliferation in a subject receiving treatment.

In a preferred embodiment, the cancer to be treated is colorectal cancer or a metastasis of colorectal cancer. In one embodiment, the cancer to be treated is pancreatic cancer or a metastasis of pancreatic cancer.

Further embodiments of the present invention relate to the use of the combination as described herein for the treatment of a subject at risk of and/or having glioma, lung, hepatocellular, gastric, ovarian, esophageal, gall bladder, renal, nasopharyngeal and/or breast cancer, or osteosarcoma.

The anti-cancer activity of niclosamide has been demonstrated in a range of human cancers. In further embodiments, the following cancers may be treated, for which niclosamide has been shown to be effective: human breast cancer, prostate cancer, colon cancer, ovarian cancer, multiple myeloma, melanoma, acute myelogenous leukemia, glioblastoma, head and neck cancer and lung cancer (as reviewed in Li et al, Cancer Lett. 2014 Jul 10; 349(1): 8-14). As summarized herein, niclosamide is able to block the Wnt signaling pathway that governs cancer initiation, progression and metastasis, thus niclosamide has a potent activity to induce cancer cell cycle arrest, growth inhibition, motility reduction and/or and apoptotic death across multiple cancer types.

In a preferred embodiment, the combination for use as a medicament as described herein is characterized in that the cancer treatment comprises treatment of a subject with a solid tumor and no detectable metastasis in one or more lymph nodes.

The treatment of patients without lymph node metastasis relates to the treatment of subjects in relatively early stages of cancer, where metastasis remains a risk to said patients.

An effective anti-tumor treatment is provided by the present invention, in addition to an effective prophylactic (or risk reduction) treatment against future metastasis, in particular in patients with early stage (such as stage 0, I or II) cancers, preferably colorectal or pancreatic cancer.

The treatment of patients with early stages of cancer using the disruption of MACC1 and S100A4 function with the combinations described herein represents a novel clinical situation. Many early stage cancer patients are not treated initially with chemotherapy. Tumor removal is generally carried out in the early stages, before chemotherapy begins. The treatment approach as described herein, in particular of patients with elevated MACC1 and/or S100A4 levels and early stage cancer, represents a therapeutic approach not previously thought possible. For example, a number of therapeutic guidelines recommend initiation of therapy with anti-proliferative drugs only during later cancer stages, for example as of stage III colorectal cancer. Until now, a need has existed in the art for means for (potentially prophylactically) treating patients with detected tumors, which have not yet metastasized. Aside from surgery, few products are available that can actively prevent or reduce the risk of cancer metastasis.

The selection of patient group and cancer stage as described herein now enables effective preventative or risk-reduction therapeutic approaches for avoiding or minimizing risk of tumor metastasis in patients with increased risk of metastasis. According to traditional protocols, clinicians would have to wait and observe whether tumor metastasis occurred after the tumor had been surgically removed before initiating further therapy. The invention now alleviates the problems inherent in previous therapeutic regimes and provides means for reducing the risk of metastasis in patients who have been diagnosed with an early stage of cancer, preferably with a solid tumor.

Until the present time, an early diagnosis of increased risk of metastasis (for example via detection of elevated MACC1 and/or S100A4 levels) could not be effectively followed up with an appropriate treatment, as no agents had been identified that could appropriately address early stage cancers with elevated MACC1 and/or S100A4 levels. Clinical practice typically would remove a tumor as quickly as possible, and not begin with additional therapies until later stages. Even in the event of detection of elevated levels of MACC1 and/or S100A4, no direct and effective therapeutic option was available for the treatment of early stage cancer patients, in order to prevent or reduce the risk of metastasis occurring, for example after removal of the tumor via surgery. The present invention solves this problem of the prior art by the provision of the MACC1 inhibitors described herein.

In a preferred embodiment the invention is characterized in that the cancer treatment according to the invention comprises treatment of a subject with stage 0, I or II colorectal cancer. In other embodiments, the invention encompasses treatment of patients with stage 0, I, IIA, IIB or IIC colorectal cancer. On the basis of the information provided herein and known guidelines, a skilled person can clearly determine which patients fall under these particular stages.

As described in more detail below, colorectal cancer staging is an accepted method for assessing cancer progression in patients and determining appropriate therapeutic approaches. Stage 0, I and II colorectal cancer patients typically show no metastasis to the lymph nodes of the subject, but may, depending on MACC1 and/or S100A4 levels, still be susceptible to developing life threatening metastasis in the future. The treatment of these patients represents a novel patient collective enabled by the MACC1 and S100A4 inhibition described herein.

In a preferred embodiment the invention is characterized in that the cancer treatment according to the invention comprises treatment of a subject with stage 0, I or II pancreatic cancer. In particular the invention envisages treatment of pancreatic cancer stages 0, IA, IB, and IIA, which are characterized by the absence of metastasis in the lymph nodes.

Treatment of stages III and IV colorectal and pancreatic cancer are also envisaged by the present invention, due to the combined anti-metastatic and anti-proliferative effects of the combination described herein.

In one embodiment, the subject of treatment will undergo and/or has undergone surgery to remove a solid tumor.

In one embodiment, the treatment and combination described herein is used in methods of treating a medical condition as a neoadjuvant. Neoadjuvant therapy represents the administration of therapeutic agents before another treatment. Neoadjuvant therapy aims to reduce the size or extent of the cancer before using radical treatment intervention, such as surgery, thus both making procedures easier and more likely to succeed and reducing the consequences of a more extensive treatment technique, which would be required if the tumor were not reduced in size or extent. In one embodiment, the combination or use thereof or treatment described herein is employed as a neoadjuvant prior to surgery to remove a tumor. In another embodiment, the combination or use thereof or treatment described herein is employed to treat and/or prevent metastatic disease prior to and/or during a surgery to remove a tumor.

As described in more detail below, pancreatic cancer staging is an accepted method for assessing cancer progression in patients and determining appropriate therapeutic approaches. On the basis of the information provided herein and known guidelines, a skilled person can clearly determine which patients fall under these particular stages.

In one embodiment of the invention the combination as described herein may be used for treating or reducing the risk of developing (metastatic) cancer, wherein said treatment comprises the treatment of a subject at risk of developing cancer, wherein said subject:
a. has been previously treated after having cancer, and preferably comprises a reduced number of cancerous cells and/or reduced cancer symptoms in comparison to before said previous treatment, and
b. comprises elevated levels of MACC1 and S100A4 in comparison to an appropriate control, for example a healthy control subject, preferably a subject with no history of cancer.

In one embodiment of the invention the combination as described herein may be used for treating or reducing the risk of developing (metastatic) cancer, wherein said treatment comprises the prophylactic treatment of cancer cell metastasis and/or cell migration in a subject at risk of developing metastases, wherein said subject:
a. has been previously treated after having colorectal cancer, and preferably comprises a reduced number of cancerous cells and/or reduced cancer symptoms in comparison to before said previous treatment, and
b. comprises elevated levels of MACC1 and S100A4 in comparison to an appropriate control, for example a healthy control subject, preferably a subject with no history of cancer.

In some embodiments of the invention a patient identified with a tumor will typically have the tumor operatively removed. The removed tumor, or the patients' blood or an appropriate sample thereof may be tested for MACC1 and/or S100A4 levels. If the patient is identified as having elevated MACC1 and/or S100A4 levels in comparison to a relevant control, the subject is an intended subject for treatment with the compounds described herein. After surgery, physicians cannot guarantee in every case that all cancerous cells have been removed. Elevated MACC1 and/or S100A4 levels in the removed tumor or in the blood of the subject indicate an increased risk of metastasis, even after surgical removal of the tumor. The administration of the combination as described herein may be carried out in order to reduce the risk of metastasis and subsequently of developing cancer re-lapse.

The present invention further relates to the combined administration of the compounds described herein with further cytostatic agents. Such agents may relate to any anti-proliferative compound known in the art.

In a preferred embodiment the MACC1-inhibitors described herein are administered to early stage cancer patients in combination with 5-FU. Fluorouracil or 5-FU (trademarked as Adrucil (i.v.), Carac or Efudex) is a drug that is a pyrimidine analog which is used in the treatment of cancer. It is a suicide inhibitor and works through irreversible inhibition of thymidylate synthase. Combined administration with the combinations described herein is associated with improved anti-proliferative and anti-metastatic effect.

Further candidates for combined therapeutic regimes relate to platinum-based antineoplastic agents, such as oxaliplatin, marketed as Eloxatin by Sanofi, which is a used in cancer chemotherapy. Oxaliplatin is used for treatment of colorectal cancer, typically along with folinic acid and 5-fluorouracil, potentially in a combination medicament. Other platinum compounds used for advanced cancers, such as cisplatin and carboplatin, may also be considered.

The invention therefore relates to methods of treatment of the cancers and patient groups described above. The invention also relates to combined methods of diagnostics, prognosis and treatment based on detection of MACC1 expression and subsequent inhibition, respectively.

The present invention further relates to a method for the treatment of cancer, or a particular cancer or group of patients as described herein, in a human subject, such as a subject with early stage cancer as described herein, comprising:
i. having a sample, such as a biological fluid, obtained from the subject,
ii. having an assay conducted on the sample, said assay comprising determining levels of MACC1 and/or S100A4 expression in the sample, for example as described for the diagnostic assays described above, and comparing the levels of MACC1 and/or S100A4 expression to a control sample, and
iii. when elevated levels of MACC1 and/or S100A4 are detected, treating the subject in order to reduce MACC1 and/or S100A4 expression and/or activity, said treatment comprising the administration of a pharmaceutical combination as described herein.

The features of the invention regarding methods of treatment and diagnosis, and descriptions of the pharmaceutical combination for use in the treatment of various medical conditions, apply to the pharmaceutical combination itself, and vice versa.

### DETAILED DESCRIPTION OF THE INVENTION

### Pharmaceutical Combination:

According to the present invention, a “pharmaceutical combination” is the combined presence of an inhibitor of the Wnt/β-catenin signaling pathway with an inhibitor of MACC1, i.e. in proximity to one another. In one embodiment, the combination is suitable for combined administration.

In one embodiment, the pharmaceutical combination as described herein is characterized in that the inhibitor of the Wnt/β-catenin signaling pathway is in a pharmaceutical composition in admixture with a pharmaceutically acceptable carrier, and the inhibitor of MACC1 is in a separate pharmaceutical composition in admixture with a pharmaceutically acceptable carrier. The pharmaceutical combination of the present invention can therefore in some embodiments relate to the presence of two separate compositions or dosage forms in proximity to each other. The agents in combination are not required to be present in a single composition.

In one embodiment, the pharmaceutical combination as described herein is characterized in that the inhibitor of the Wnt/β-catenin signaling pathway and the inhibitor of MACC1 according to any one of the preceding claims are present in a kit, in spatial proximity but in separate containers and/or compositions. The production of a kit lies within the abilities of a skilled person. In one embodiment, separate compositions comprising two separate agents may be packaged and marketed together as a combination. In other embodiments, the offering of the two agents in combination, such as in a single catalogue, but in separate packaging is understood as a combination.

In one embodiment, the pharmaceutical combination as described herein is characterized in that the inhibitor of the Wnt/β-catenin signaling pathway and the inhibitor of MACC1 according to any one of the preceding claims are combined in a single pharmaceutical composition in admixture with a pharmaceutically acceptable carrier. Combination preparations or compositions are known to a skilled person, who is capable of assessing compatible carrier materials and formulation forms suitable for both agents in the combination.

### Inhibitors:

According to the present invention, an “inhibitor” in the context of “an inhibitor of the Wnt/β-catenin signaling pathway” or “an inhibitor of MACC1” is considered any agent, substance, compound, molecule or other means leading to a slowing, repressing, blocking or other interfering or negative action on the activity, function, expression of or signaling caused by the named target.

For example, an inhibitor of MACC1 may affect MACC1 protein function, MACC1 expression (transcription or translation) and/or MACC1-mediated signaling, either directly or indirectly. For example, an inhibitor of the Wnt/β-catenin signaling pathway may affect Wnt/β-catenin signaling, or any factor involved in Wnt/β-catenin signaling either directly or indirectly. The inhibitors as described herein may also be termed “agents”. References to the “agents” in the context of the combinations and methods described herein are to be understood as the inhibitor of the Wnt/β-catenin signaling pathway and the inhibitor of MACC1. Preferred inhibitors are those described herein.

### Synergy:

To determine or quantify the degree of synergy or antagonism obtained by any given combination, a number of models may be employed. Typically, synergy is considered an effect of a magnitude beyond the sum of two known effects. In some embodiments, the combination response is compared against the expected combination response, under the assumption of non-interaction calculated using a reference model (refer Tang J. et al. (2015) What is synergy? The saariselkä agreement revisited. Front. Pharmacol., 6, 181).

Commonly-utilized reference models include the Highest single agent (HSA) model (Berenbaum M.C. (1989) What is synergy. Pharmacol. Rev., 41, 93-141), the Loewe additivity model (Loewe S. (1953) The problem of synergism and antagonism of combined drugs. Arzneimiettel Forschung, 3, 286-290), the Bliss independence model (Bliss C. I. (1939) The toxicity of poisons applied jointly. Ann. Appl. Biol., 26, 585-615.), and more recently, the Zero interaction potency (ZIP) model (Yadav B. et al. (2015) Searching for drug synergy in complex dose-response landscapes using an interaction potency model. Comput. Struct. Biotechnol. J., 13, 504-505). The assumptions being made in these reference models are different from each other, which may produce somewhat inconsistent conclusions about the degree of synergy. Nevertheless, according to the present invention, when any one of these models indicates synergy between the agents in the combination as described herein, it may be assumed synergy has been achieved. Preferably, 2, 3 or all 4 of these models will reveal synergy between any two agents of the combination described herein.

Without limitation, four reference models are preferred, which can produce reliable results: (i) HSA model, where the synergy score quantifies the excess over the highest single drug response; (ii) Loewe model, where the synergy score quantifies the excess over the expected response if the two drugs are the same compound; (iii) Bliss model, where the expected response is a multiplicative effect as if the two drugs act independently; and (iv) ZIP model, where the expected response corresponds to an additive effect as if the two drugs do not affect the potency of each other.

The most widely used combination reference, and preferred model for determining synergy, is “Loewe additivity”, or the “Loewe model” (Loewe (1928), Ergebn. Physiol. 27:47-187; Loewe and Muischnek. “Effect of combinations: mathematical basis of the problem” Arch. Exp. Pathol. Pharmakol. 114: 313-326, 1926; Loewe S. (1953) The problem of synergism and antagonism of combined drugs. Arzneimittel Forschung, 3, 286-290), or “dose additivity” which describes the trade-off in potency between two agents when both sides of a dose matrix contain the same compound. For example, if 50% inhibition is achieved separately by 1 uM of drug A or 1 uM of drug B, a combination of 0.5 uM of A and 0.5 uM of B should also inhibit by 50%. Synergy over this level is especially important when justifying the clinical use of proposed combination therapies, as it defines the point at which the combination can provide additional benefit over simply increasing the dose of either agent.

As a further example of determining Loewe Additivity (or dose additivity), let d₁ and d₂ be doses of compounds 1 and 2 producing in combination an effect e. We denote by Dₑ₁ and Dₑ₂ the doses of compounds 1 and 2 required to produce effect e alone (assuming these conditions uniquely define them, i.e. that the individual dose-response functions are bijective). dₑ₁/Dₑ₂ quantifies the potency of compound 1 relatively to that of compound 2. d₂Dₑ₁/Dₑ₂ can be interpreted as the dose of compound 2 converted into the corresponding dose of compound 1 after accounting for difference in potency. Loewe additivity is defined as the situation where d₁+d₂Dₑ₁/Dₑ₂=Dₑ₁ or d₁/Dₑ₁+d2/Dₑ₂=1. Geometrically, Loewe additivity is the situation where isoboles are segments joining the points (Dₑ₁, 0) and (0, Dₑ₂) in the domain (d₁, d₂). If we denote by f₁(d₁), f₂(d₂) and the dose-response functions of compound 1, compound 2 and of the mixture respectively, then dose additivity holds when d₁/f₁⁻¹ (f₁₂ (d₁, d₂))+d₂/f₂⁻¹ (f₁₂ (d₁, d₂))=1.

### Combined Administration:

According to the present invention, the term “combined administration”, otherwise known as co-administration or joint treatment, encompasses in some embodiments the administration of separate formulations of the compounds described herein, whereby treatment may occur within minutes of each other, in the same hour, on the same day, in the same week or in the same month as one another. Alternating administration of two agents is considered as one embodiment of combined administration. Staggered administration is encompassed by the term combined administration, whereby one agent may be administered, followed by the later administration of a second agent, optionally followed by administration of the first agent, again, and so forth. Simultaneous administration of multiple agents is considered as one embodiment of combined administration. Simultaneous administration encompasses in some embodiments, for example the taking of multiple compositions comprising the multiple agents at the same time, e.g. orally by ingesting separate tablets simultaneously. A combination medicament, such as a single formulation comprising multiple agents disclosed herein, and optionally additional anti-cancer medicaments, may also be used in order to co-administer the various components in a single administration or dosage.

A combined therapy or combined administration of one agent may precede or follow treatment with the other agent to be combined, by intervals ranging from minutes to weeks. In embodiments where the second agent and the first agent are administered separately, one would generally ensure that a significant period of time did not expire between the time of each delivery, such that the first and second agents would still be able to exert an advantageously combined synergistic effect on a treatment site. In such instances, it is contemplated that one would contact the subject with both modalities within about 12-24 h of each other and, more preferably, within about 6-12 h of each other, with a delay time of only about 12 h being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

In the meaning of the invention, any form of administration of the multiple agents described herein is encompassed by combined administration, such that a beneficial additional therapeutic effect, preferably a synergistic effect, is achieved through the combined administration of the two agents.

### MACC1:

Metastasis remains a major challenge in colorectal cancer (CRC) management. One recent key finding in understanding molecular pathogenesis of CRC metastasis is the identification of the gene MACC1. MACC1 has been discovered as prognostic biomarker for metastasis and metastasis-free survival in CRC. Meanwhile MACC1 is confirmed as a decisive driver for tumorigenesis and metastasis in various other solid cancers.

“MACC1” as used herein refers to the metastasis associated in colon cancer 1 gene in Homo sapiens according to the Gene ID: 346389 of the NCBI database. The gene encodes an 852 amino acid protein recorded under Gen Bank ID: AAI37091. Appropriate means for detecting MACC1 encoding nucleic acids or proteins are provided in the examples below.

Inhibitors of MACC1 can be determined using established and routine techniques. For example, any given assay may be employed where a substance suspected of being a MACC1 inhibitor is assessed and compared to an appropriate control, using the commercially available technologies described herein (e.g. ELISA kits available from Aviva Systems Biology OKCD09378 or Biomatik EKU05926) or the technology of the cited prior art (e.g. Juneja, Kobelt et al. PLoS Biol. 2017 Jun 1;15(6)). MACC1 protein or activity may be assessed or levels of MACC1 transcripts may be determined in a cellular assay or from a biological sample, in order to determine reduced activity or amounts of MACC1 induced by treatment of any given substance (candidate inhibitor).

### MEK Inhibitors:

In some embodiments, the MACC1 inhibitor is a MEK inhibitor, preferably a MEK1 inhibitor. A MEK inhibitor is any substance that inhibits a mitogen-activated protein kinase enzyme, preferably MEK1 and/or MEK2. These inhibitors affect the MAPK/ERK pathway, which is overactive in some cancers.

The mitogen-activated protein kinase (MAPK) signaling pathways involve a family of protein kinases that play critical roles in regulation of diverse cellular activities, including cell proliferation, survival, differentiation, motility, and angiogenesis. The MAPK pathways transduce signals from various extracellular stimuli (growth factors, hormones, cytokines and environmental stresses), leading to distinct intracellular responses via a series of phosphorylation events and protein-protein interactions. MEK proteins belong to a family of enzymes that lie upstream to their specific MAPK targets in each of the four MAP kinase signaling pathways. Multiple MEK enzymes have been identified. These MEK enzymes selectively phosphorylate serine/threonine and tyrosine residues within the activation loop of their specific MAP kinase substrates. MEK1 and MEK2 are closely related. They participate in the Ras/Raf/MEK/ERK signal transduction cascade. MEK 1, also designated as MAPKK-1, is the prototype member of MEK family proteins. It is encoded by the gene MAP2K1 located on chromosome 15q22.31. The gene, MAP2K2, encoding MEK 2 protein, resides on chromosome 19p13.3. MEK 1/2 proteins consist of a N-terminal sequence, a protein kinase domain, and a C-terminal sequence (as reviewed in Akinleye et al, J Hematol Oncol. 2013; 6: 27).

A number of MEK inhibitors are known in the art. A skilled person is capable of identifying a substance of this established class using standard methods or literature resources. MEK inhibitors presently in clinical development include, for example, CI-1040 (PD184352), PD0325901,selumetinib (AZD6244), MEK162, AZD8330, TAK-733, GDC-0623, refametinib (RDEA119; BAY 869766), pimasertib (AS703026), R04987655 (CH4987655), R05126766, WX-554, R04987655 (CH4987655), GDC-0973 (XL518), AZD8330 and HL-085 (as reviewed in Cheng and Tian, Molecules 2017, 22, 1551, and in Akinleye et al, J Hematol Oncol. 2013; 6: 27).

MEK inhibitors are commercially available and can be sourced as required, selected from the group, without limitation, consisting of selumetinib (AZD6244), trametinib (GSK1120212), PD0325901, U0126, PD184352 (CI-1040), PD98059, BIX 02189, pimasertib (AS-703026), BIX 02188, TAK-733, AZD8330, binimetinib (MEK162, ARRY-162, ARRY-438162), PD318088, Honokiol, SL-327, refametinib (RDEA119, Bay 86-9766), myricetin, BI-847325, cobimetinib (GDC-0973, RG7420), GDC-0623 and APS-2-79 HCl. Further MEK inhibitors in development relate to LNP-3794, SHR-7390, CKI-27, CS-3006 and E-6201. In one embodiment, the MEK inhibitor is not U0126.

### Statins:

Statins, also known as HMG-CoA reductase inhibitors, are a class of lipid-lowering medications. They reduce cardiovascular disease and mortality in those who are at high risk of cardiovascular disease. Statins are effective in lowering LDL cholesterol and statins are therefore widely used for primary prevention in people at high risk of cardiovascular disease, as well as in secondary prevention for those who have developed cardiovascular disease. Statins are commercially available and include, without limitation, atorvastatin (Lipitor), fluvastatin (Lescol, Lescol XL), lovastatin (Mevacor, Altoprev), pravastatin (Pravachol), rosuvastatin (Crestor), simvastatin (Zocor), and pitavastatin (Livalo).

### Determination of MACC1 Levels in Patients:

The determination of elevated MACC1 levels in patient samples has been described in the art. These methods may be applied in the present invention.

Examples of detecting MACC1 levels from blood plasma are disclosed in Burock S, Herrmann P, Wendler I, Niederstrasser M, Wernecke K D, Stein U. “Circulating MACC1 transcripts in gastric cancer patient plasma as diagnostic and prognostic biomarker”, World J Gastroenterol, accepted 22.7.2014. Briefly, the authors tested differences between groups in terms of MACC1 transcript levels in plasma by using non-parametric (exact) Wilcoxon- Mann-Whitney tests (because of deviations of the distributions from normality and small samples). Samples obtained from tumor-free volunteers were compared with those from patients with primary tumors without and with synchronous organ metastases, patients with metachronous metastases, and follow-up patients.

Furthermore, samples obtained from patients with tumors vs. those with tumors and metastases were compared. Results are expressed as median (range) or frequencies (%). p<0.05 was considered to be significant. For evaluating the diagnostic value of circulating MACC1 transcripts in plasma of cancer patients, the authors calculated sensitivity and specificity with a fourfold table for those cancer patients, who were newly diagnosed with a primary tumor without or with synchronous metastases compared to the blood samples of tumor-free volunteers. The authors used Kaplan Meier curves in combination with log rank test for survival analyses of newly diagnosed and of all gastric patients. The respective cut-off values for the biomarkers were the median of the investigated groups (primary diagnosis or all patients).

Further examples of methods for detecting elevated MACC1 levels are described in Stein U, Burock S, Herrmann P, Wendler I, Niederstrasser M, Wernecke K W, Schlag P M, “Circulating MACC1 transcripts in colorectal cancer patient plasma predict metastasis and prognosis”, PLoS ONE, 7:e49249, 2012. Differences between groups in terms of MACC1 transcript levels in plasma were tested by using non-parametric Wilcoxon-Mann-Whitney tests (dependent on the distribution of normality): tumor-free volunteers vs. patients with primary tumors without and with synchronous metastases, to patients with metachronous metastases, and to follow-up patients; patients with tumors vs. those with tumors and metastases. In case of small samples, greater differences in sample sizes, large but unbalanced groups, data sets containing ties, or sparse data, tests were carried out in an exact version. P<0.05 was considered to be significant. To define the diagnostic value of circulating MACC1 transcripts in plasma, sensitivity and specificity were calculated with a fourfold table for colorectal cancer (test- and validation-set), colon, and rectal cancer patients, who were newly diagnosed with a primary tumor without or with synchronous metastases compared to the blood samples of tumor-free volunteers. In accordance with their chronological examination and initial blood taking patients were grouped in a test-set and validation-set. A test-set of newly diagnosed CRC patients is employed, the optimal cut-off value of MACC1 determined (sensitivity 75%, specificity 76%), and this cut-off value applied for the validation-set of CRC patients. For survival of newly diagnosed and all cancer patients, Kaplan Meier curves in combination with log rank test were used. The cut-off value of MACC1 was the median of investigated groups (primary diagnosis or all patients).

Examples of testing for and determining elevated levels in tumor tissue are disclosed in Stein U, Walther W, Arlt F, Schwabe H, Smith J, Fichtner I, Birchmeier W, Schlag P M, “MACC1, a newly identified key regulator of HGF/Met signaling, predicts colon cancer metastasis”, Nature Med 15:59-67, 2009. The authors evaluated statistical significance with the nonparametric two-sided Mann-Whitney rank-sum test. The authors used ROC analysis and Mann-Whitney tests for comparison of non-metastasized and metastasized cases. The authors evaluated correlation between MACC1 and MET in subjects by using the nonparametric Spearman-Rho test. The authors evaluated Kaplan-Meier curves with the log-rank test. The authors used logistic and Cox regression for evaluation of MACC1 as an independent metastasis marker. Data represent means ±s.d.

Further examples of testing for and determining elevated MACC1 levels in tumor tissue are disclosed in Nitsche U, Rosenberg R, Balmert A, Schuster T, Slotta-Huspenina J, Herrmann P, Bader F G, Friess H, Schlag P M, Stein U, Janssen K P, “Integrative marker analysis allows risk assessment for metastasis in stage II colon cancer”, Ann Surgery, 256: 763-771, 2012. Recurrence-free survival (i.e., distant metastasis-free survival) was considered as primary endpoint. Statistical evaluation was performed using IBM® SPSS® Statistics Version 19 (SPSS Inc., IBM Corporation, Somers, New York, USA). In order to derive optimal cut-off values of gene expression levels, maximally selected log-rank statistics performed by R Software version 2.13.0 (R Foundation for Statistical Computing, Vienna, Austria) were used. To consider multiple test issue within these analyses, the R-function maxstat.test was employed. Time-dependent survival probabilities were estimated with the Kaplan-Meier method, and the log-rank test was used to compare independent subgroups. To investigate the effect on survival of multivariable relationships among covariates, Cox proportional hazard models were used. Multivariable analysis of binary outcome data was assessed by logistic regression. Area-under-curve (AUC) values were calculated by time-dependent receiver operating-characteristic (ROC) analyses for censored survival data. Recurrence-free survival times as well as estimated hazard ratios (HRs) were calculated and reported in 95% confidence intervals (95% Cls). Clustering of the patients into different groups was performed by the SPSS® Two Step Cluster analysis function. A post-hoc power analysis using N-Query Software revealed that with the total number of distant-recurrent cases, hazard ratios of ≥3.2 were detectable with a type-2 error 20% (power 80%) at a two-sided level of significance of 5%, when using a log-rank test. All statistical tests were performed two-sided, and p-values less than 0.05 were considered to be statistically significant. No correction of p-values was applied to adjust for multiple test issue.

Wnt/β-catenin Signaling Pathway:

As used herein, the terms “Wnt/β-catenin signaling pathway” and “Wnt-signaling” or “Wnt-signaling pathway” may be used interchangeably.

Colorectal cancer is often associated with activation of the Wnt/β-catenin signaling pathway and high expression of the metastasis-inducing gene S100A4. In addition to its function in the cell-cell adhesion, β-catenin is also an important mediator of the canonical Wnt signalling pathway. When no Wnt signalling occurs, two scaffolding proteins, the tumor suppressor APC and AXIN, form the so-called destruction complex with β-catenin, which facilitates the sequential phosphorylation of β-catenin by CKI and GSK3-β at the amino-terminus. The phosphorylations recruit the F box/WD repeat protein β-TrCP-containing E3 ubiquitin ligase, which marks β-catenin for proteasomal degradation. The Wnt signalling pathway is activated by binding of the Wnt ligand to the Frizzled transmembrane receptor, a serpentine receptor with an amino-terminal cysteine-rich domain. The complex then interacts with a single-pass transmembrane protein of the LDL receptor family (LRP5/6). It is not clear how the FRZ/LRP complex regulates the kinase activity of the destruction complex. However, it is suggested that the activity axin/GSK3-β is inhibited by a mechanism involving the interaction of axin with LRP5/6 or the action of the axin binding molecule Dishevelled (DSH). The unphosphorylated β-catenin translocates to the nucleus where it binds to the amino-terminus of the Tcf/Lef (T cell factor/lymphoid enhancer factor) family of DNA-binding proteins and activates the transcription of target genes. The Tcf/Lef proteins repress target genes in the absence of β-catenin, but transform into transcriptional activators once they bind to β-catenin.

The relevance of the Wnt pathway for cancer cells is indicated by the high percentage of mutations occurring in the genes of the Wnt pathway. For example, over 90% of colorectal cancers bear mutations that result in Wnt pathway activation. These mutations generally affect β-catenin phosphorylation and stability, hindering its degradation via the ubiquitin pathway. Non-phosphorylated β-catenin accumulates in the cytoplasm, is transported to the nucleus, and interacts with TCF family transcription factors to control target genes. Nuclear β-catenin accumulation has been associated with late stages of tumor progression and development of metastases, and the presence of mutated β-catenin is associated with aggressive tumor growth and poor prognosis.

As one example, S100A4 is considered a marker molecule or read-out for Wnt/β-catenin signaling. Other read-out molecules, by which their levels are indicative of Wnt/β-catenin signaling inhibition, are, without limitation, c-myc, MMP-7, -9 or -13.

Wnt/beta-Catenin Signaling and Small Molecule Inhibitors have been described in detail in Voronkov et al (Current Pharmaceutical Design, 2013, 19, 634-664).

Additional inhibitors of the Wnt/β-catenin signaling pathway relate to XAV939, IWR1, IWP-1, IWP-2, JW74, JW55, okadaic acid, tautomycin, SB239063, SB203580, ADP-HPD, 2-[4-(4-fluorophenyl)piperazin-1-yl]-6-methylpyrimidin4(3H)-one, PJ34, cambinol, sulindac, 3289-8625, J01-017a, NSC668036, filipin, IC261, PF670462, bosutinib, PHA665752, imatinib, ICG-001, ethacrynic acid, PKF115-584, PNU-74654, PKF118-744, CGP049090, PKF118-310, ZTM000990, BC21, GDC-0941, Rp-8-Br-cAMP.

Inhibitors of the Wnt/β-catenin signaling pathway can be determined using established and routine techniques. For example, QIAGEN provides a broad range of assay technologies for assessing WNT signaling research that enables analysis of gene expression and regulation, epigenetic modification, genotyping, and signal transduction pathway activation. Any given assay may be employed where a substance suspected of being a Wnt/β-catenin signaling pathway inhibitor is assessed and compared to an appropriate control, using the commercially available technologies described herein or the technology of the cited prior art (e.g. Sack et al. Mol Biol Cell. 2011 Sep;22(18):3344-54; Sack et al., J Natl Cancer Inst. 2011 Jul 6;103(13):1018-36).

### S100A4:

One major target that is linked to metastasis formation is S100 calcium binding protein A4 (5100A4), an 11 kDa protein, originally identified as metastasin 1(MTS1). S100A4 is also known as: 18A2, 42A, CAPL, FSP1, MTS1, P9KA, PEL98. “S100A4” as used herein refers to the S100 calcium binding protein A4 in Homo sapiens according to the protein ID: NP_002952 of the NCBI database. The S100A4 gene encodes a 101 amino acid protein recorded under Gene ID: 6275. Appropriate means for detecting S100A4 encoding nucleic acids or S100A4 proteins are provided herein.

S100A4 is overexpressed in many different types of cancer such as gallbladder, bladder, breast, esophageal, gastric, pancreatic, hepatocellular, non-small cell lung and especially colorectal cancer. Increased expression of S100A4 is strongly associated with aggressiveness of a tumor, its ability to metastasize and poor survival in patients. However, S100A4 itself is not tumorigenic because transgenic mice overexpressing S100A4 do not develop tumors per se. But, when S100A4 transgenic mice are crossed with mice demonstrating spontaneous tumor formation, it leads to aggressive tumor growth and metastasis. Moreover, S100A4-null mice injected with highly metastatic mouse mammary carcinoma cells show no metastases. These observations suggest that S100A4 is essential for the process of metastasis formation.

S100A4 plays a major role in cellular processes such as migration, invasion, adhesion and angiogenesis, which form the basis for metastasis formation. For instance, S100A4 increases cell motility by interacting with proteins from the cytoskeleton, such as the heavy chain of non-muscle myosin II (MYH9). Moreover, S100A4 participates in cell adhesion by interaction with protein tyrosine phosphatase receptor type F (PTPRF) interacting protein, binding protein 1 (PPFIBPI; also known as liprin β-1) and promotes cell invasion and angiogenesis via upregulation of metallomatrix peptidase (MMPs).

Inhibitors of S100A4 can be determined using established and routine techniques. For example, any given assay may be employed where a substance suspected of being a S100A4 inhibitor is assessed and compared to an appropriate control, using the commercially available technologies described herein (e.g. ELISA kits available from Aviva Systems Biology OKCA02438 or LifeSpan Biolsciences LS-F9180-1) or the technology of the cited prior art (e.g. Sack et al., J Natl Cancer Inst. 2011 Jul 6;103(13):1018-36; Garrett et al, Biochemistry. 2008 Jan 22; 47(3): 986-996.). S100A4 protein or activity may be assessed or levels of S100A4 transcripts may be determined in a cellular assay or from a biological sample, in order to determine reduced activity or amounts of S100A4 induced by treatment of any given substance (candidate inhibitor).

### Determination of S100A4 Levels in Patients:

The determination of elevated S100A4 levels in patient samples has been described in the art. These methods may be applied in the present invention.

In a preferred embodiment the invention relates to the treatment of a group of patients that are identified by increased expression of S100A4, preferably whereby the cancerous cells show increased expression of S100A4. S100A4 expression can be determined using any commonly known method in the art, such as RT-PCR for analysis of increased expression of S100A4 transcript or immunological based methods such as western blot or ELISA for detection of increased expression of S100A4 protein, or any other diagnostic tools that can provide relative measurements of S100A4 expression in comparison to “normal” or “healthy” or “low risk” cells.

As is disclosed in US20140294957A1, total RNA can be isolated from 4×10⁵ cells plated in a 6-well-plates 24 hour before cells are lyzed with Trizol Reagent (Invitrogen). Alternatively, RNA can be isolated from blood samples, or tumor tissue samples, and treated accordingly. For example, RNA is extracted with Trizol RNA extraction reagent (Invitrogen) according to the manufacturer's instructions. Quantification of the RNA concentration is performed with Nanodrop (Peqlab) and 50 ng total RNA is reverse transcribed with random hexamers in 10 mM MgCl2; 1×RT-buffer, 250 μM pooled dNTPs, 1 U per μL RNAse inhibitor and 2.5 U per μL MuLV reverse transcriptase (all Applied Biosystems). Reaction occurrs at 42° C. for 15 minutes, 99° C. for 5 minutes and subsequent cooling at 5° C. for 5 minutes. The cDNA product is amplified in a total volume of 10 μL in 96-well-plates in the LightCycler 480 (Roche) using the following conditions: 95° C., 10 minutes, followed by 45 cycles of 95° C. for 10 seconds, 61° C. for 30 seconds and 72° C. for 4 seconds, with appropriate primers, such as those disclosed in US20140294957A1, which is hereby incorporated in entirety by reference. For cDNA quantification of the housekeeping gene glucose-6-phosphate dehydrogenase (G6PD) the hG6PDH Roche Kit (Roche Diagnostics, Mannheim, Germany) is used according to standard instructions. Data analysis is performed with e.g. LightCycler® 480 Software release 1.5.0 SP3. For each qRT-PCR reaction a mean of the duplicate is calculated. Each mean value of the expressed gene is normalized to the respective mean amount of the G6PD cDNA.

Additional Diagnostic Approaches in Determining S100A4 and/or MACC1 Levels:

The invention further relates to kits, the use of the kits and methods for determining S100A4 and/or MACC1 levels. Such means can also be used to determine S100A4 and/or MACC1 levels during functional testing of inhibitors. The herein provided definitions, e.g. provided in relation to the methods, also apply to the kits of the invention. In particular, the invention relates to the use of kits for determining S100A4 and/or MACC1 levels, wherein said kit comprises
detection reagents for determining the level of S100A4 and/or MACC1, and
reference data for determining elevated levels of S100A4 and/or MACC1, in particular reference data for threshold or cut-off value(s), wherein said reference data is preferably stored on a computer readable medium and/or employed in the form of computer executable code configured for comparing the determined levels of S100A4 and/or MACC1 with the threshold or cut-off value(s).

As used herein, “reference data” comprise reference level(s) of S100A4 and/or MACC1, preferably from a healthy patient. The levels of S100A4 and/or MACC1 in the sample of the subject can be compared to the reference levels comprised in the reference data of the kit. The reference data can also include a reference sample to which the level of S100A4 and/or MACC1 is compared. The reference data can also include an instruction manual how to use the kits of the invention.

The kit may additionally comprise items useful for obtaining a sample, such as a blood sample, for example the kit may comprise a container, wherein said container comprises a device for attachment of said container to a canula or syringe, is a syringe suitable for blood isolation, exhibits an internal pressure less than atmospheric pressure, such as is suitable for drawing a pre-determined volume of sample into said container, and/or comprises additionally detergents, chaotropic salts, ribonuclease inhibitors, chelating agents, RNAse inhibitor proteins, and mixtures thereof.

As used herein, the “detection reagent” or the like are reagents that are suitable to determine the herein described marker(s), e.g. of S100A4 and/or MACC1. Such exemplary detection reagents are, for example, nucleic acid primers for amplifying S100A4 and/or MACC1 encoding nucleic acid sequences. Such primers might be used in a nucleic acid amplification reaction, e.g. a PCR method. Such exemplary detection reagents are, for example, ligands, e.g. antibodies or fragments thereof, which specifically bind to the S100A4 and/or MACC1 peptide or epitopes of the herein described marker(s). Such ligands might be used in immunoassays.

The term “nucleic acid amplification reaction” refers to any method comprising an enzymatic reaction, which allows the amplification of nucleic acids. One preferred embodiment of the invention relates to a polymerase chain reaction (PCR). Another preferred embodiment relates to real time PCR (RT-PCR) or quantitative RT-PCR (qRT-PCR), as it allows the quantification of the amplified target in real-time. The term “real-time PCR” is intended to mean any amplification technique which makes it possible to monitor the progress of an ongoing amplification reaction as it occurs (i.e. in real time). Data is therefore collected during the exponential phase of the PCR reaction, rather than at the end point as in conventional PCR. Measuring the kinetics of the reaction the early phases of PCR provides distinct advantages over traditional PCR detection. In real-time PCR, reactions are characterized by the point in time during cycling when amplification of a target is first detected rather than the amount of target accumulated after a fixed number of cycles. The higher the starting copy number of the nucleic acid target, the sooner a significant increase in fluorescence is observed. Traditional PCR methods may also be applied, and use separation methods, such as agarose gels, for detection of PCR amplification at the final phase of or end-point of the PCR reaction. For qRT-PCR no post-PCR processing of the unknown DNA sample is necessary as the quantification occurs in real-time during the reaction. Furthermore, an increase in reporter fluorescent signal is directly proportional to the number of amplicons generated. A skilled person is aware of the reagents and methods necessary for carrying out such detection.

### Cancer:

The invention relates to pharmaceutical combinations for MACC1 inhibition and Wnt/β-catenin signaling inhibition for the treatment of cancer or cancer-like disorders, such as a cell proliferative disorder or cancer metastasis. The terms “cancer”, “proliferative disorder” or “cellular proliferative disorder” may be understood interchangeably and refer to any disorder in which the proliferative capabilities of the affected cells is different from the normal proliferative capabilities of unaffected cells. An example of a cell proliferative disorder is neoplasia. Malignant cells develop as a result of a multistep process. The term “cancer”, tumor disease” or “malignant tumor” may refer to a tumor or hematopoietic disease no longer under normal cellular growth control. The term “cancerous cell” includes a cell afflicted by any one of the cancerous conditions provided herein. The term “carcinoma” refers to a malignant new growth made up of epithelial cells tending to infiltrate surrounding tissues, and to give rise to metastases.

A cell proliferative disorder as described herein may be a neoplasm, commonly referred to as a tumor or tumor. Such neoplasms are either benign or malignant. The term “neoplasm”, “tumor” or “tumor” refers to a new, abnormal growth of cells or a growth of abnormal cells that reproduce faster than normal. A neoplasm creates an unstructured mass (a tumor) which can be either benign or malignant. The term “benign” refers to a tumor that is noncancerous, e.g. its cells do not proliferate or invade surrounding tissues.

In another aspect, the invention provides a method of preventing, treating, and/or managing a solid tumor in a patient, the method comprising administering to a patient in need thereof a prophylactically effective regimen or a therapeutically effective regimen, the regimen comprising administering to the patient a compound of the invention, or a pharmaceutically acceptable salt thereof, wherein the patient has been diagnosed with a solid tumor. In particular embodiments of this aspect, the solid tumor is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney-cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, or retinoblastoma.

### Metastasis:

In one embodiment, the invention relates to the pharmaceutical combination, or the two agents of the combination, for use in the treatment and/or prophylaxis of tumor metastasis, preferably by reducing cellular motility of cancerous cells. As used herein, the term “metastasis” refers to the spread of cancer cells from the place where they first formed to another part of the body.

Metastasis may be considered a multistep process where tumor cells disseminate from the primary tumor and colonize distant organs. The steps include, without limitation, tumor cell invasion of basement membranes and the surrounding tissue, intravasation into blood vessels, survival there, extravasation and/or growth at different organ sites. To achieve these steps, precise coordination of cell movement and matrix remodeling are required and can be interrupted by an anti-metastatic agent. Determining an effect against cancer metastasis may therefore employ determining an inhibitory effect against one or more of cell invasion, i.e. invasion of basement membranes and the surrounding tissue, cell motility or cell movement, invasion into a blood or lymphatic vessel (intravasation), and/or leakage of cancer cells from a blood or lymphatic vessel (extravasation). According to the present invention reduced cell motility is preferably assessed.

Models for assessing an effect against metastasis are disclosed herein and are known to a person skilled in the art. The in vitro approach employed preferably uses a wound healing assay with the HCT116 human colon cancer cell line. An effect on metastasis is quantified based on the direct effect on reduced cell motility of the HCT116 cells observed in the wound healing assay.

Effects may also be observed for reducing cancer cell motility, migration and/or invasion. In some embodiments, cancer metastasis can be distinguished from cancer proliferation, thereby representing a distinct medical use. In some embodiments, cell proliferation and cell metastasis are both reduced.

As used herein “cell migration” or “motility” is intended to mean movement of a cell towards a target, such as a chemical or physical signal. Cells often migrate in response to external signals, including chemical signals and mechanical signals. Cancer cells are capable of migrating, and exhibit cell motility, which are characteristics in metastatic cancer and may therefore be targeted to treat and/or reduce or reduce the risk of metastasis. Chemotaxis is one example of cell migration or motility regarding response to a stimulus. In vitro chemotaxis assays such as Boyden chamber assays may be used to determine whether cell migration occurs for any given cell.

For example, cancer cells may be purified and analysed. Chemotaxis assays (for example according to Falk et al., 1980 J. Immuno. Methods 33:239-247) can be performed using plates where a particular chemical signal is positioned with respect to the cells of interest and the transmigrated cells then collected and analyzed. For example, Boyden chamber assays entail the use of chambers isolated by filters, used as tools for accurate determination of chemotactic behavior. The pioneer type of these chambers was constructed by Boyden (Boyden (1962) “The chemotactic effect of mixtures of antibody and antigen on polymorphonuclear leucocytes”. J Exp Med 115 (3): 453). The motile cells are placed into the upper chamber, while fluid containing the test substance is filled into the lower one. The size of the motile cells to be investigated determines the pore size of the filter; it is essential to choose a diameter which allows active transmigration. For modelling in vivo conditions, several protocols prefer coverage of filter with molecules of extracellular matrix (collagen, elastin etc.) Efficiency of the measurements can be increased by development of multiwell chambers (e.g. NeuroProbe), where 24, 96, 384 samples are evaluated in parallel. Advantage of this variant is that several parallels are assayed in identical conditions. Methods such as this can be used to assess whether any given cancer cell type exhibits cell motility or migration, i.e. a tumor samples could be obtained, the cells cultured and assayed in such a method. Furthermore, any given inhibitor can be assessed for its effect on cell motility using an assay such as this. The effect of any given combination as described herein may be assessed to determine a synergistic effect in affected migration, which is a read-out on efficacy in treating metastasis.

### Cancer Staging:

A staging system is a standardized way in which clinicians describe the extent of a cancer. The most commonly used staging system for colorectal cancer is that of the American Joint Committee on Cancer (AJCC), sometimes also known as the TNM system (refer American Cancer Society for details).

The TNM system describes 3 key pieces of information: T describes how far the main (primary) tumor has grown into the wall of the intestine and whether it has grown into nearby areas; N describes the extent of spread to nearby (regional) lymph nodes. Lymph nodes are small bean-shaped collections of immune system cells that are important in fighting infections; M indicates whether the cancer has spread (metastasized) to other organs of the body.

### Colorectal Cancer Staging:

Colorectal cancer can spread almost anywhere in the body, but the most common sites of spread are the liver and lungs. Numbers or letters appear after T, N, and M to provide more details about each of these factors. The numbers 0 through 4 indicate increasing severity. The letter X means “cannot be assessed because the information is not available.”

T categories of colorectal cancer describe the extent of spread through the layers that form the wall of the colon and rectum. Tx: No description of the tumor's extent is possible because of incomplete information; Tis: The cancer is in the earliest stage (in situ). It involves only the mucosa. It has not grown beyond the muscularis mucosa (inner muscle layer); T1: The cancer has grown through the muscularis mucosa and extends into the submucosa; T2: The cancer has grown through the submucosa and extends into the muscularis propria (thick outer muscle layer); T3: The cancer has grown through the muscularis propria and into the outermost layers of the colon or rectum but not through them. It has not reached any nearby organs or tissues; T4a: The cancer has grown through the serosa (also known as the visceral peritoneum), the outermost lining of the intestines; T4b: The cancer has grown through the wall of the colon or rectum and is attached to or invades into nearby tissues or organs.

N categories indicate whether or not the cancer has spread to nearby lymph nodes and, if so, how many lymph nodes are involved. To get an accurate idea about lymph node involvement, most doctors recommend that at least 12 lymph nodes be removed during surgery and looked at under a microscope. Nx: No description of lymph node involvement is possible because of incomplete information; N0: No cancer in nearby lymph nodes; N1: Cancer cells are found in or near 1 to 3 nearby lymph nodes; N1a: Cancer cells are found in 1 nearby lymph node; N1b: Cancer cells are found in 2 to 3 nearby lymph nodes; N1c: Small deposits of cancer cells are found in areas of fat near lymph nodes, but not in the lymph nodes themselves; N2: Cancer cells are found in 4 or more nearby lymph nodes; N2a: Cancer cells are found in 4 to 6 nearby lymph nodes; N2b: Cancer cells are found in 7 or more nearby lymph nodes.

M categories indicate whether or not the cancer has spread (metastasized) to distant organs, such as the liver, lungs, or distant lymph nodes. MO: No distant spread is seen; M1a: The cancer has spread to 1 distant organ or set of distant lymph nodes; M1b: The cancer has spread to more than 1 distant organ or set of distant lymph nodes, or it has spread to distant parts of the peritoneum (the lining of the abdominal cavity).

Once a person's T, N, and M categories have been determined, usually after surgery, this information is combined in a process called stage grouping. The stage is expressed in Roman numerals from stage I (the least advanced) to stage IV (the most advanced). Some stages are subdivided with letters.

Stage 0: Tis, N0, M0: The cancer is in the earliest stage. It has not grown beyond the inner layer (mucosa) of the colon or rectum. This stage is also known as carcinoma in situ or intramucosal carcinoma.

Stage I: T1-T2, N0, M0: The cancer has grown through the muscularis mucosa into the submucosa (T1) or it may also have grown into the muscularis propria (T2). It has not spread to nearby lymph nodes or distant sites.

Stage IIA: T3, N0, M0: The cancer has grown into the outermost layers of the colon or rectum but has not gone through them (T3). It has not reached nearby organs. It has not yet spread to the nearby lymph nodes or distant sites.

Stage IIB: T4a, N0, M0: The cancer has grown through the wall of the colon or rectum but has not grown into other nearby tissues or organs (T4a). It has not yet spread to the nearby lymph nodes or distant sites.

Stage IIC: T4b, N0, M0: The cancer has grown through the wall of the colon or rectum and is attached to or has grown into other nearby tissues or organs (T4b). It has not yet spread to the nearby lymph nodes or distant sites.

Stage IIIA: One of the following applies: T1-T2, N1, M0: The cancer has grown through the mucosa into the submucosa (T1) and it may also have grown into the muscularis propria (T2). It has spread to 1 to 3 nearby lymph nodes (N1a/N1b) or into areas of fat near the lymph nodes but not the nodes themselves (N1c). It has not spread to distant sites; or T1, N2a, M0: The cancer has grown through the mucosa into the submucosa (T1). It has spread to 4 to 6 nearby lymph nodes (N2a). It has not spread to distant sites.

Stage IIIB: One of the following applies: T3-T4a, N1, M0: The cancer has grown into the outermost layers of the colon or rectum (T3) or through the visceral peritoneum (T4a) but has not reached nearby organs. It has spread to 1 to 3 nearby lymph nodes (N1a/N1b) or into areas of fat near the lymph nodes but not the nodes themselves (N1c). It has not spread to distant sites; T2-T3, N2a, M0: The cancer has grown into the muscularis propria (T2) or into the outermost layers of the colon or rectum (T3). It has spread to 4 to 6 nearby lymph nodes (N2a). It has not spread to distant sites; or T1-T2, N2b, M0: The cancer has grown through the mucosa into the submucosa (T1) or it may also have grown into the muscularis propria (T2). It has spread to 7 or more nearby lymph nodes (N2b). It has not spread to distant sites.

Stage IIIC: One of the following applies: T4a, N2a, M0: The cancer has grown through the wall of the colon or rectum (including the visceral peritoneum) but has not reached nearby organs (T4a). It has spread to 4 to 6 nearby lymph nodes (N2a). It has not spread to distant sites; T3-T4a, N2b, M0: The cancer has grown into the outermost layers of the colon or rectum (T3) or through the visceral peritoneum (T4a) but has not reached nearby organs. It has spread to 7 or more nearby lymph nodes (N2b). It has not spread to distant sites; or T4b, N1-N2, M0: The cancer has grown through the wall of the colon or rectum and is attached to or has grown into other nearby tissues or organs (T4b). It has spread to at least one nearby lymph node or into areas of fat near the lymph nodes (N1 or N2). It has not spread to distant sites.

Stage IVA: Any T, Any N, M1a: The cancer may or may not have grown through the wall of the colon or rectum, and it may or may not have spread to nearby lymph nodes. It has spread to 1 distant organ (such as the liver or lung) or set of lymph nodes (M1a).

Stage IVB: Any T, Any N, M1b: The cancer may or may not have grown through the wall of the colon or rectum, and it may or may not have spread to nearby lymph nodes. It has spread to more than 1 distant organ (such as the liver or lung) or set of lymph nodes, or it has spread to distant parts of the peritoneum (the lining of the abdominal cavity) (M1b).

### Pancreatic Cancer Staging:

T categories: TX: The main tumor cannot be assessed; T0: No evidence of a primary tumor; Tis: Carcinoma in situ (the tumor is confined to the top layers of pancreatic duct cells). Very few pancreatic tumors are found at this stage; T1: The cancer is still within the pancreas and is 2 centimeters (cm) (about ¾ inch) or less across; T2: The cancer is still within the pancreas but is larger than 2 cm across; T3: The cancer has grown outside the pancreas into nearby surrounding tissues but not into major blood vessels or nerves; T4: The cancer has grown beyond the pancreas into nearby large blood vessels or nerves.

N categories: NX: Nearby (regional) lymph nodes cannot be assessed; N0: The cancer has not spread to nearby lymph nodes; N1: The cancer has spread to nearby lymph nodes.

M categories: M0: The cancer has not spread to distant lymph nodes (other than those near the pancreas) or to distant organs such as the liver, lungs, brain, etc; M1: The cancer has spread to distant lymph nodes or to distant organs;

Stage grouping for pancreatic cancer: Once the T, N, and M categories have been determined, this information is combined to assign an overall stage of 0, I, II, Ill, or IV (sometimes followed by a letter). This process is called stage grouping.

Stage 0 (Tis, N0, M0): The tumor is confined to the top layers of pancreatic duct cells and has not invaded deeper tissues. It has not spread outside of the pancreas. These tumors are sometimes referred to as pancreatic carcinoma in situ or pancreatic intraepithelial neoplasia III (Panin III).

Stage IA (T1, N0, M0): The tumor is confined to the pancreas and is 2 cm across or smaller (T1). It has not spread to nearby lymph nodes (N0) or distant sites (M0).

Stage IB (T2, N0, M0): The tumor is confined to the pancreas and is larger than 2 cm across (T2). It has not spread to nearby lymph nodes (N0) or distant sites (M0).

Stage IIA (T3, N0, M0): The tumor is growing outside the pancreas but not into major blood vessels or nerves (T3). It has not spread to nearby lymph nodes (N0) or distant sites (M0).

Stage IIB (T1-3, N1, M0): The tumor is either confined to the pancreas or growing outside the pancreas but not into major blood vessels or nerves (T1-T3). It has spread to nearby lymph nodes (N1) but not to distant sites (M0).

Stage III (T4, Any N, M0): The tumor is growing outside the pancreas into nearby major blood vessels or nerves (T4). It may or may not have spread to nearby lymph nodes (Any N). It has not spread to distant sites (M0).

Stage IV (Any T, Any N, M1): The cancer has spread to distant sites (M1).

### Treatment:

In the present invention “treatment” or “therapy” generally means to obtain a desired pharmacological effect and/or physiological effect. The effect may be prophylactic in view of completely or partially preventing a disease and/or a symptom, for example by reducing the risk of a subject having a particular disease or symptom, or may be therapeutic in view of partially or completely curing a disease and/or adverse effect of the disease.

In the present invention, “therapy” includes arbitrary treatments of diseases or conditions in mammals, in particular, humans, for example, the following treatments (a) to (c): (a) Prevention of onset of a disease, condition or symptom in a patient; (b) Inhibition of a symptom of a condition, that is, prevention of progression of the symptom; (c) Amelioration of a symptom of a condition, that is, induction of regression of the disease or symptom.

In particular, the treatment described herein relates to either reducing or inhibiting tumour growth via reducing or inhibiting proliferation of cancerous cells, or reducing the metastatic properties of cancerous cells. The prophylactic therapy as described herein is intended to encompass prevention or reduction of risk of developing metastatic cancer, due to a reduced likelihood of cancerous cells to metastasize after treatment with the compounds described herein.

### Pharmaceutical Compositions and Methods of Administration:

The present invention also relates to a pharmaceutical composition comprising the compounds described herein.

The invention also relates to pharmaceutically acceptable salts of the compounds described herein, in addition to enantiomers and/or tautomers of the compounds described.

The term “pharmaceutical composition” refers to a combination of the agent as described herein with a pharmaceutically acceptable carrier. The phrase “pharmaceutically-acceptable” refers to molecular entities and compositions that do not produce a severe allergic or similar untoward reaction when administered to a human. As used herein, “carrier” or “carrier substance” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions.

The pharmaceutical composition containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions. Tablets contain the active ingredient in admixture with non-toxic pharmaceutically acceptable excipients which are suitable for the manufacture of tablets. The tablets may be uncoated or they may be coated by known techniques to delay disintegration and absorption in the gastrointestinal tract and thereby provide a sustained action over a longer period.

Dosage levels of the order of from about 0.01 mg to about 500 mg per kilogram of body weight per day are useful in the treatment of the indicated conditions. For example, a cancer may be effectively treated by the administration of from about 0.01 to 50 mg of the inventive molecule per kilogram of body weight per day (about 0.5 mg to about 5 g per patient per day). The amount of active ingredient that may be combined with the carrier materials to produce a single dosage form will vary depending upon the host treated and the particular mode of administration. For example, a formulation intended for the oral administration of humans may vary from about 5 to about 95% of the total composition. Dosage unit forms will generally contain between from about 1 mg to about 5000 mg of active ingredient. It will be understood, however, that the specific dose level for any particular patient will depend upon a variety of factors including the activity of the specific compound employed, the age, body weight, general health, sex, diet time of administration, route of administration, rate of excretion, drug combination and the severity of the particular disease undergoing therapy. The dosage effective amount of compounds according to the invention will vary depending upon factors including the particular compound, toxicity, and inhibitory activity, the condition treated, and whether the compound is administered alone or with other therapies.

The invention relates also to a process or a method for the treatment of the mentioned pathological conditions. The compounds of the present invention can be administered prophylactically or therapeutically, preferably in an amount that is effective against the mentioned disorders, to a warm-blooded animal, for example a human, requiring such treatment, the compounds preferably being used in the form of pharmaceutical compositions.

In some embodiments, a patient may receive therapy for the treatment and/or management of the cancer before, during or after the administration of the therapeutically effective regimen of the compound of the invention, or a pharmaceutically acceptable salt thereof. Non-limiting examples of such a therapy include chemotherapy, radioimmunotherapy, toxin therapy, prodrug-activating enzyme therapy, antibody therapy, surgical therapy, immunotherapy, radiation therapy, targeted therapy (i.e. therapy directed toward a specific target or pathway, e.g. tyrosine kinase, etc.), and any combination thereof. In some embodiments, the patient has not previously received a therapy for the treatment and/or management of the cancer.

### FIGURES

The invention is further described by the figures. These are not intended to limit the scope of the invention.

FIG. 1: Statins inhibit MACC1 expression

HCT116 cells were treated with increasing amounts (1-10 μM) of different, clinically relevant statins dissolved in DMSO. Following 24 h of treatment cells were harvested for RNA and protein isolation. MACC1 mRNA expression was analyzed by qRT-PCR and is expressed relative to DMSO treated control samples. Protein amounts were analyzed by western blot. All tested statins were able to significantly reduce MACC1 mRNA expression and MACC1 protein in a dose-dependent fashion.

FIG. 2: Migration induced by supernatants from MACC1 overexpressing cells.

Cell culture supernatants from SW480/vector (low MACC1 expression), SW480/MACC1 (different clones, ectopically MACC1 overexpressing) and SW620 (endogenously high MACC1 expression) were collected to treat different tumor cell lines in vitro. SW480 cells (low MACC1 expression) show increased migration in the boyden chamber assay when treated with cell culture supernatants of MACC1 positive cells compared to controls treated with supernatants of MACC1 low SW480 cells. SW620 cells (high endogenous MACC1 expression) show no increase in migration under these conditions. When MACC1 is depleted by shRNA in these cells, motility is decreased and can be rescued by cell culture supernatants of MACC1 positive cells. These results were confirmed in another human tumor cell line of another entity (MiaPaCa, pancreas carcinoma). Therefore, supernatants collected from MACC1 overexpressing cells induce migration on different wild type cells without ectopic MACC1 overexpression.

FIG. 3: SILAC-Mass-spec analysis

Cell cultures of SW480/vector (low MACC1 expression) and SW480/MACC1 (ectopically high MACC1 expression) were treated with light and heavy media to label newly synthesized proteins secreted in the cell culture supernatant. After protein isolation and LC-MS/MS analysis the MACC1 induced secretome was identified. The MACC1 specific supernatants harbor newly secreted S100A4. SW480/vector vs SW480/MACC1: MACC1 overexpression induces S100 proteins, in particular S100A4 secretion.

FIG. 4: Biomarker combination of MACC1 AND S100A4 identifies best colorectal cancer patients at high risk

Tumor (A) and plasma (B) samples were collected from colorectal cancer patients and analyzed for MACC1 and S100A4 gene expression by qRT-PCR. Patients with low gene expression of both biomarkers show the best metastasis-free and overall survival. Patients with high gene expression of one biomarker show reduced metastasis-free and overall survival. The worst metastasis-free and overall survival was evident for patients with high gene expression of both biomarkers. Therefore, MACC1 and S100A4 can identify patients with increased risk for metastasis formation compared to patients with low expression. This prognosis is further improved if both biomarkers are highly expressed: (A) in primary CRC tumors; (B) in CRC patient plasma.

FIG. 5: MACC1 enhances Wnt signaling.

MACC1 overexpression in SW480 (A) and HCT116 (B) cells increases, while MACC1 depletion in SW620 (C) cells decreases, TCF signaling activity, indicative for positive regulation of Wnt signaling by MACC1. Immunoblot of MACC1 overexpression in SW480 and knockdown in SW620 (D). β-catenin knockdown decreases MACC1-induced TCF promoter activity (E) and MACC1-induced migration (F), indicating that MACC1 induces Wnt signaling through β-catenin in colorectal cancer cells.

FIG. 6: MACC1 induces S100A4 expression through Wnt signaling.

Ectopic overexpression of MACC1 in HCT116 cells (A) increases TCF promoter activity (B), subsequently upregulating S100A4-promoter activity (C) and S100A4 gene expression at mRNA (D) as well as protein (E) level. Knockout of MACC1 in SW620 cells (F) decreases TCF promoter activity (G), subsequently downregulating S100A4-promoter activity (H) and S100A4 gene expression at mRNA (I) as well as protein (J) level. In a colorectal cancer cohort of 54 patients MACC1 and S100A4 mRNA expression level were positively correlated (K).

FIG. 7: The effect of statins and niclosamide applied as single agents on cellular motility and proliferation in vitro.

HCT116 cells were treated with three concentrations of atorvastatin, lovastatin, fluvastatin and niclosamide for 48 h. Wound closure was monitored every second hour using the IncuCyte real-time system. Wound closure was measured relative to the initial wound and expressed as fold change compared to DMSO treated samples. A pre-established image collection of HCT116 was used to teach the software detection of cells and wound. The experiments show, that there is a dose dependent reduction of wound closure ability for all drugs tested. Untreated and DMSO treated cells served as controls. Data represents mean ±SEM of at least 3 independent experiments in triplicate.

FIG. 8: The effect of statins and niclosamide applied as combined agents on cellular motility and proliferation in vitro.

HCT116 cells were treated with three concentrations of atorvastatin, lovastatin, fluvastatin in combination with three concentrations of niclosamide for 48 h. As a representative example the combination of a 50% reduced dose is shown. For each drug combination the synergy matrix for all concentrations is depicted. Positive values demonstrate the presence of synergy between the two agents. A negative value would indicate antagonism between the two agents. A value of 0 indicates an additive effect of two agents.

Wound closure was monitored every second hour using the IncuCyte real-time system. Wound closure was measured relative to the initial wound and expressed as fold change compared to DMSO treated samples. A pre-established image collection of HCT116 was used to teach the software detection of cells and wound. The experiments show that there is a synergistic reduction of wound closure when a statin is combined with niclosamide. Untreated and DMSO treated cells served as controls. Data represents mean ±SEM of at least 3 independent experiments in triplicate.

FIG. 9: The effect of MEK1 inhibitors AZD6244 and GSK1120212 and niclosamide applied as combined agents on cellular motility and proliferation in vitro.

HCT116 cells were treated with three concentrations of GSK1120212 in combination with three concentrations of niclosamide. For each drug combination the synergy matrix for all concentrations is depicted. Positive values demonstrate the presence of synergy between the two agents. A negative value would indicate antagonism between the two agents. A value of 0 indicates an additive effect of two agents.

A synergistic example of the combination of 0.1 μM GSK1120212 and 0.25 μM niclosamide is shown. For treatments the synergy matrix for all concentrations is depicted. Wound closure was monitored every second hour using the IncuCyte real-time system. Wound closure was measured relative to the initial wound and expressed as fold change compared to DMSO treated samples. A pre-established image collection of HCT116 was used to teach the software detection of cells and wound. The experiments show that there is a synergistic reduction of wound closure when the MEK1 inhibitor GSK1120212 (trametinib) is combined with niclosamide. Untreated and DMSO treated cells served as controls.

FIG. 10: The effect of atorvastatin, fluvastatin and niclosamide in an in vivo mouse model at the equivalent of maximum human dose.

Severe combined immunodeficiency (SCID)-beige mice (n=10 per group) were intrasplenically transplanted with HCT116-CMVp-Luc cells and treated daily with solvent, 13 mg/kg atorvastatin or fluvastatin (p.o.), 328 mg/kg niclosamide (p.o.) or the combination of niclosamide with one of the statins, at the indicated doses. These doses correspond to the maximum human doses established for the statins and niclosamide. Metastasis to the liver was analyzed with bioluminescence imaging after an intraperitoneal application of 150 mg/kg D-Luciferin at the experimental endpoint. Following whole animal imaging livers were removed and analyzed as isolated organs. Metastasis formation was significantly reduced when atorvastatin, fluvastatin and niclosamide were applied as single drugs. This metastasis inhibition was also evident in the combinatorial treatment.

FIG. 11: The effect of atorvastatin, fluvastatin and niclosamide in an in vivo mouse model at reduced dose.

Severe combined immunodeficiency (SCID)-beige mice (n=10 per group) were intrasplenically transplanted with HCT116-CMVp-Luc cells and treated daily with solvent, 1.5 mg/kg atorvastatin or fluvastatin (p.o.), 164 mg/kg niclosamide (p.o.) or the combination of niclosamide with one of the statins, at the indicated doses. These doses therefore correspond to 12.5% of the maximum human dose for the statins and 50% of the maximum human dose for niclosamide. Metastasis to the liver was analyzed with bioluminescence imaging after an intraperitoneal application of 150 mg/kg D-Luciferin at the experimental endpoint. Following whole animal imaging livers were removed and analyzed as isolated organs. No reduction in metastasis formation was observed when atorvastatin, fluvastatin and niclosamide were applied as single drugs. However, a reduction in metastasis was observed in the combinatorial treatments, further supporting that the combination of active agents leads to a synergistic effect.

### EXAMPLES

The invention is further described by the following examples. These are not intended to limit the scope of the invention.

### Materials and Methods

### Cell Lines and Drug Treatment

HCT116, SW620 and SW480 colorectal carcinoma cells were purchased from ATCC. HCT116 cells were stably transduced with GFP/ MACC1-GFP plasmids to produce MACC1 overexpressing cell lines and SW620 cells were stably transduced by sh-control/sh-MACC1 plasmids to produce MACC1 knockdown cell lines. SW480 stable MACC1 overexpressing cell lines were created using pCDNA3.1 MACC1 plasmids and the control cells were produced using pCDNA3.1 empty plasmids followed clone selection using Gentamycin (Gibco). Knock-out of MACC1 in SW620 cells was achieved by CRISPR-Cas9 based gene editing followed by Puromycin (Gibco) based selection and subsequently the clones were picked using single cell sorting. Cells were cultured in RPMI supplemented with 10% FCS in 5% CO₂ humidified atmosphere and sub passaged 2-3 times a week to maintain cultures subconfluent. Drugs were solubilized in DMSO, stock concentrations were 2 mM for niclosamide (Sigma-Aldrich, St Louis, Mo., USA), 2 mM for GSK1120212 (trametinib, Selleck Chemicals, Munich, Germany) and selumetinib (AZD6244, Selleck Chemicals, Munich, Germany) and 10 mM for statins (atorvastatin, lovastatin, fluvastatin, TCI Deutschland GmbH, Eschborn, Germany). All drugs were pre-diluted to 1000× final concentration in DMSO. Control cells were treated with equal amounts of DMSO.

### Reporter Assays

Luciferase reporter assay was used to assess Wnt signaling and S100A4-promoter activity. Wnt signaling activity was measured using TOP-flash reporter construct containing 6×TCF promoter consensus sequence cloned ahead of firefly luciferase in a pGL4.23 reporter plasmid (Promega) (Kindly provided by Dr. Giridhar Mudduluru). S100A4 promoter activity was measured using S100A4 promoter luciferase system harboring core S100A4 promoter sequence cloned ahead of firefly luciferase reporter in a pGL1.4 reporter plasmid (Invitrogen). Briefly, 2×10⁵ cells were seeded into 24-well plates. 24 h post incubation cells were transfected with 500 ng of the reporter luciferase construct along with 25 ng of renilla luciferase plasmid to normalize for transfection efficiency. Transfection was carried out using Lipofectamine 2000 (Life Technologies) transfection reagent according to the manufacturers instructions. After 48 h of transfection luciferase activity was measured using the Dual Luciferase Assay Kit (Promega). The firefly luciferase activity values were normalized using renilla luciferase values and data were represented as Mean±S.E.M of three independent experiments.

### RNA Interference

Silencer Select pre-designed siRNA against β-catenin (#16704) and negative control (#4390844) were purchased from Ambion. Transfection of the cells was carried out using Lipofectamine RNAiMAX (Life Technologies) transfection reagent according to the manufacturers instructions. After 48h, cells were re-seeded for further treatments and/or analysis.

### Boyden Chamber Migration Assay

Cells were starved in FCS-free medium overnight. Transwell Boyden chamber inserts (Corning) were equilibrated in medium containing 10% FCS for 4 h at 37° C. prior to cell seeding. 1×10⁵ serum-starved cells were seeded into each insert in medium containing 2% FCS. Medium containing 10% FCS was added to the bottom well as a chemoattractant and chambers were incubated at 37° C. in humidified incubator at 5% CO₂. After 16 h cells on the bottom end of the transwell membrane were harvested by trypsinization. The migrated cells were quantified using Cell-Titer-Glo reagent (Promega) according to manufacturer's instructions.

RNA Isolation and qRT-PCR

RNA was isolated using the Gene Matrix Universal RNA Purification Kit (EURx, Poland) according to the manufacturer's instructions. RNA was quantified and 50 ng of RNA was reverse transcribed using a reaction mix containing MuLV reverse transcriptase, 10 mM MgCl2, 1× PCR-Buffer, 250 μM pooled dNTPs, 1U RNAse inhibitor and random hexamers (all from Applied Biosystems). The reaction was performed at 42° C. for 15 min, 99° C. for 5 min and subsequent cooling at 4° C. for 5 min. The cDNA was amplified using SYBR Green chemistry (Promega) using the LightCycler 480 II system (Roche Diagnostics) at the following PCR conditions: 95° C. for 2 min followed by 45 cycles of 95° C. for 7 s, 60° C. for 10 s and 72° C. for 20 s. The primers used for quantification are as follows: MACC1 Fwd 5′-TTCTTTTGATTCCTCCGGTGA-3′ and Rev 5′-ACTCTGATGGGCATGTGCTG-3′; S100A4 Fwd 5′-TGTGATGGTGTCCACCTTCC-3′ and Rev 5′- CCTGTTGCTGTCCAAGTTGC-3′. Data analysis was performed with LightCycler 480 Software release 1.5.0 SP3 (Roche Diagnostics). Mean values was calculated from RT-qPCR duplicates. Each mean value of the expressed gene was normalized to the respective RP-II cDNA. Data is represented as mean ±S.E.M of three independent experiments.

### Protein Extraction and Immunoblotting

Whole cell protein extraction was performed on 5×10⁵ HCT116 and SW620 cells. Cells were lysed using RIPA buffer (50 mM Tris-HCT pH 7.5, 150 mM NaCl, 1% Nonidet P-40) supplemented with cOmplete Protease inhibitor (Roche) for 30 min at 4° C. The lysate was clarified and the protein content was measured using the BCA Kit (Pierce). 20 μg protein lysate were denatured with LDS-containing NuPage Loading buffer (invitrogen) and DTT for 10 min.

Proteins were then resolved in 10% SDS-PAGE followed by protein transfer onto PVDF membranes (BioRAD). The membrane was blocked with 5% w/v skim milk powder and 1% w/v BSA in TBST (10 mM Tris-HCL pH 8, 150 mM NaCl and 0.1% Tween 20) for 60 min. The membrane was incubated overnight with the respective primary antibodies (anti-MACC1, #HPA020081, Sigma Aldrich; S100A4, #5114, Dako and β-Actin, #31430, Invitrogen; Lamin B1, #12586s, Cell Signalling). The membranes were washed and further incubated with corresponding horseradish peroxidase-tagged secondary antibodies (anti-mouse IgG HRP-conjugated, #31430, Invitrogen and anti-rabbit IgG HRP-conjugated, #W4018, Promega) for 60 min. The membranes were further washed and developed using chemiluminescent reagent WesternBright ECL kit (Advansta) and subsequent exposure to Fuji medical X-ray film SuperRX (Fujifilm).

### Wound Healing Assay and Calculation of Synergy

For wound healing assays the HCT116 human colon cancer cell line was used. HCT116 cells are endogenously positive for MACC1 and S100A4 expression at the mRNA and protein level. In addition, they show decreased motility when either marker is inhibited.

For wound healing (scratch) experiments cells were passaged to a density of 6×10⁵ per ml and cultured for 48 h. Cells were then harvested and counted. For wound healing assays 1.2×10⁵ cells were seeded in 100 μl RPMI supplemented with 10% FCS in 96 well Image Lock Plates (Essen Bioscience, Hertfordshire, UK). Cells were allowed to adhere for 6 hours. Wounds in the monolayer were applied using the IncuCyte WoundMaker (Essen Bioscience). After applying the wounds 100 μl of 2-fold concentrated drug solutions were added. DMSO treated and untreated samples served as controls. For each drug three different (niclosamide 1 μM, 0.5 μM and 0.25 μM, each statin 5 μM, 2.5 μM and 1.25 μM, MEK1 inhibitors 1 μM, 0.1 μM and 0.01 μM) concentrations and combinations thereof were applied, each in triplicate. Of all statins, we tested lovastatin, fluvastatin and atorvastatin. Wound closure was monitored every second hour in the IncuCyte system (Essen Bioscience). A pre-established image collection of HCT116 was used to teach the software detection of cells and wound. Wound confluency was expressed relative to DMSO treated controls.

Synergy was analyzed using combenefit 2.021. DMSO treated samples were set to 100%. To calculate synergy, we used the Loewe isobole equation model. For the calculations dose-response curves for three single concentrations and the nine combinations thereof were used.

The Loewe model was applied since there is a certain degree of target interaction already shown: first, active Wnt-signaling is important for MACC1 driving tumor progression and invasiveness (Lemos C, Clin Cancer Res, 2016) and second, S100A4 is a Wnt-signaling target gene and is found in the MACC1 induced secretome. The three single concentrations and nine combinations thereof were expressed relative to the control.

Animals and Drug Treatment in vivo

All experiments were performed in accordance with the United Kingdom Coordinated Committee on Cancer Research (UKCCCR) guidelines and approved by the responsible local authorities (State Office of Health and Social Affairs, Berlin, Germany). For in vivo drug testing a xenograft mouse model was used as described earlier (Stein 2009 Nat Med, Sack 2011 J Natl Cancer Inst,

Juneja & Kobelt 2017 Plos Biol). In brief, HCT116-CMVp-LUC cells (5×10⁵ cells per mouse, resuspended in 50 μL PBS) were intrasplenically transplanted into 6-week-old female SCID beige (SCID bg/bg) mice. The animals were assigned randomly into treatment groups.

Drugs were administered as a suspension using a gavage tube. Both niclosamide (either 328 mg/kg or 164 mg/kg) and a statin (atorvastatin or fluvastatin, at 13 mg/kg or 1.5 mg/kg) were administered daily, orally. Control mice were treated with the appropriate volume of solvent solution. The in vivo experimentation and luminescence imaging were conducted as described below.

The in vivo experiments were terminated when the animals in the control group showed signs of increased suffering due to tumor/metastasis burden and liver damage like swollen abdomen (ascites formation), reduced activity, and reduced food intake (ethical/humane endpoint).

For luminescence imaging, mice were anesthetized with 5% Isoflurane and received intraperitoneally 150 mg/kg D-luciferin (Biosynth, Staad, Switzerland) dissolved in sterile PBS. Anesthesia was maintained with 2% isoflurane. Imaging was performed with the NightOWL LB 981 system (Berthold Technologies, Bad Wildbad, Germany). ImageJ version 1.48v (NIH, Bethesda, Md.) was used for color coding of signal intensity (presenting a 256 grayscale) and overlay pictures.

### Results

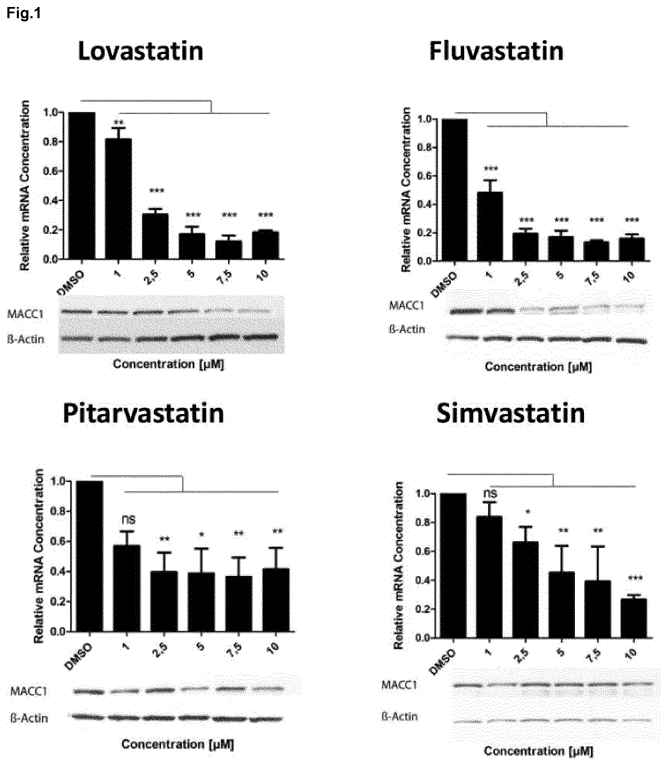

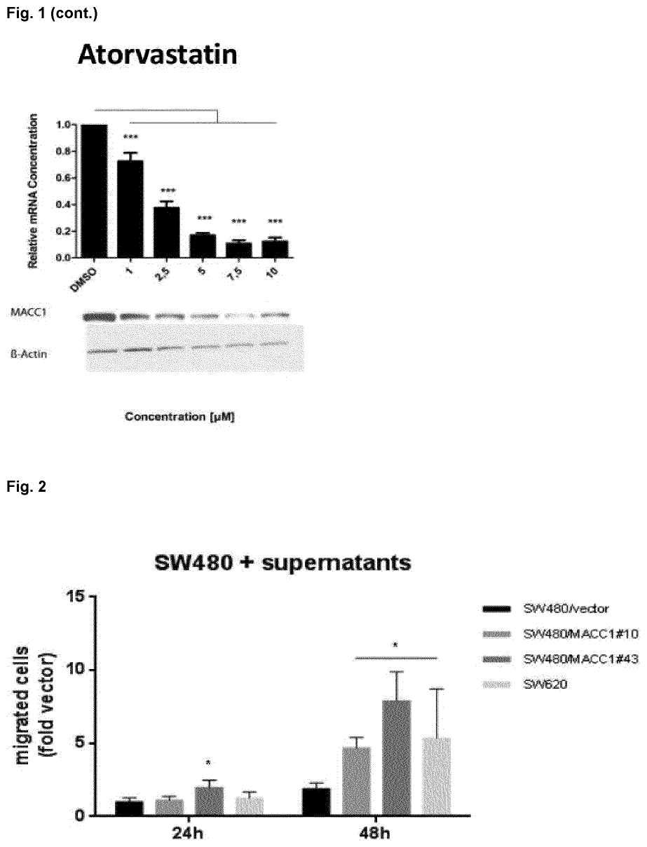

MACC1 was reported to enhance WNT signaling and its target gene expression leading to increased cancer cell migration and invasion in vitro and tumor formation as well as metastasis in vivo (Stein et al. 2009 Nat Med. Jan;15(1):59-67; Zhen et al. 2014, Oncotarget. Jun 15;5(11):3756-69; Lemos et al. 2016, Clin Cancer Res. Jun 1;22(11):2812-24). Ectopic overexpression of MACC1 increased β-catenin nuclear localization, thereby increasing WNT target genes. Conversely, MACC1 knock-down decreased β-catenin nuclear localization and reduced their expression. To inhibit MACC1 gene expression, different clinically relevant statins were tested for their ability to reduce MACC1 mRNA and protein expression. All tested statins reduced MACC1 gene expression significantly (FIG. 1).

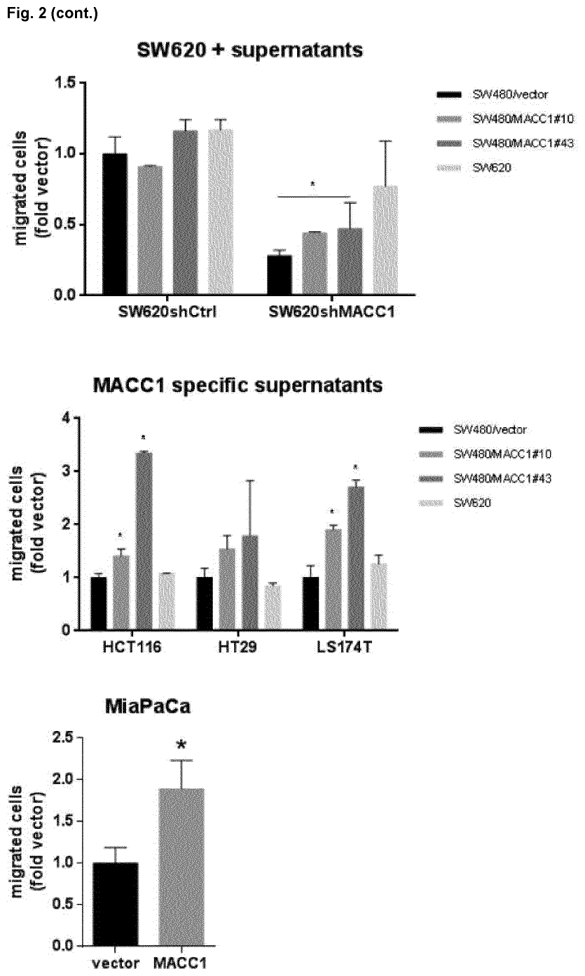

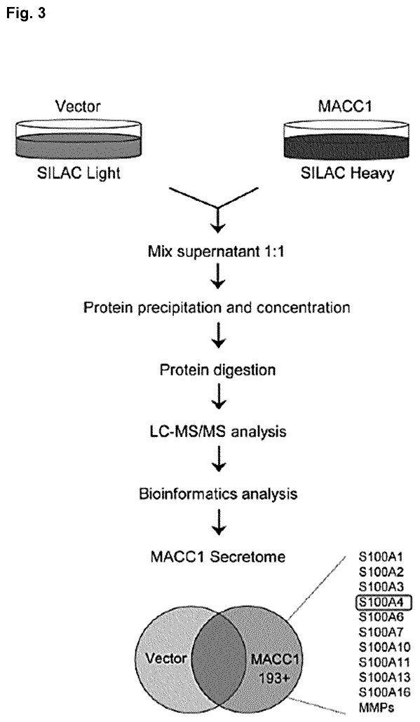

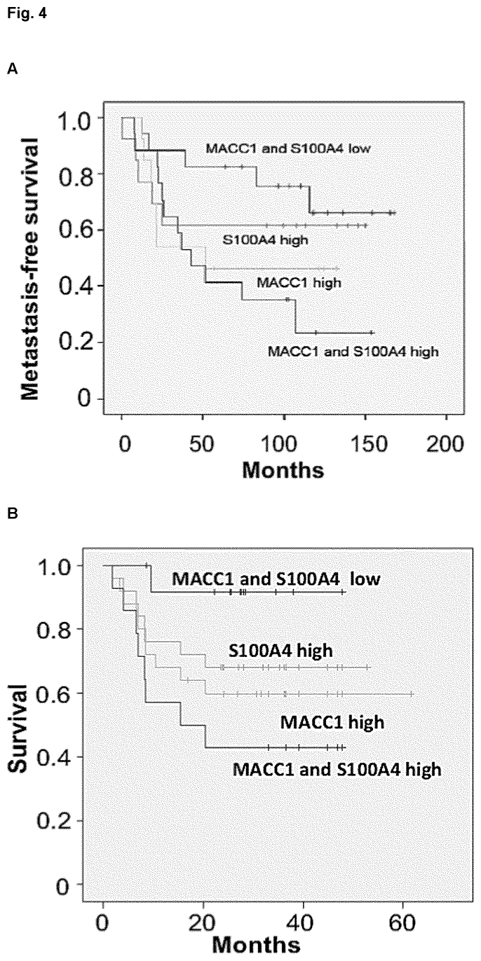

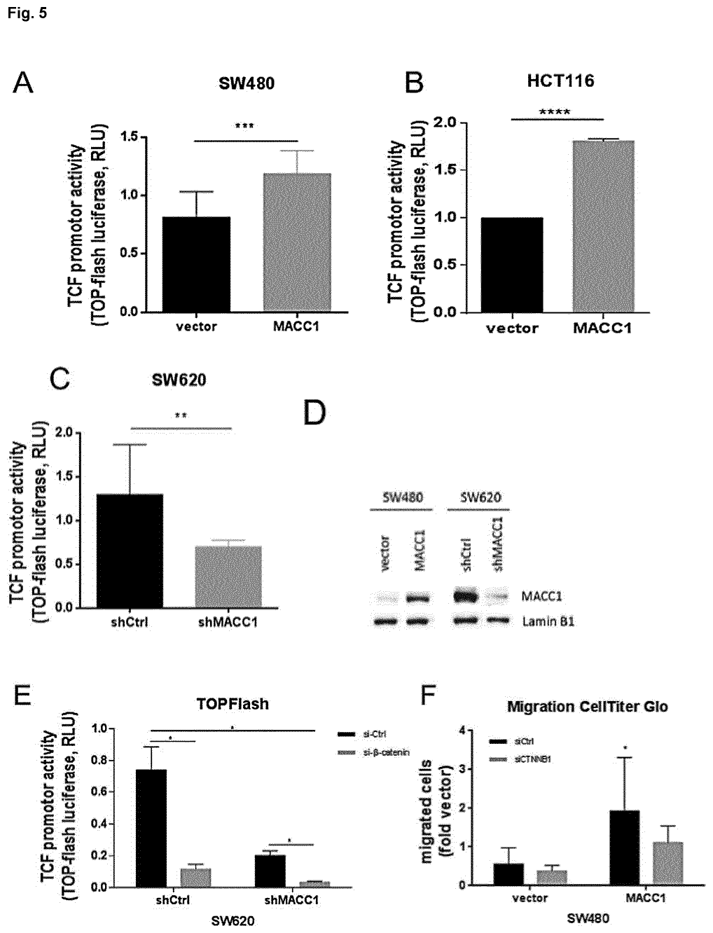

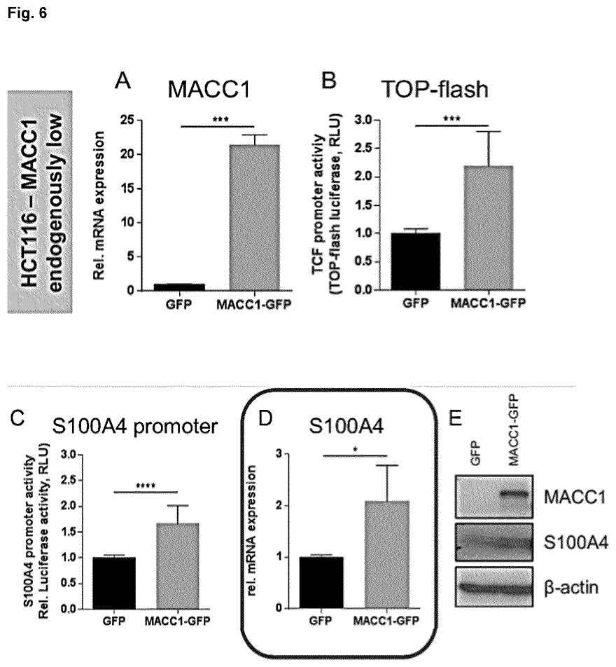

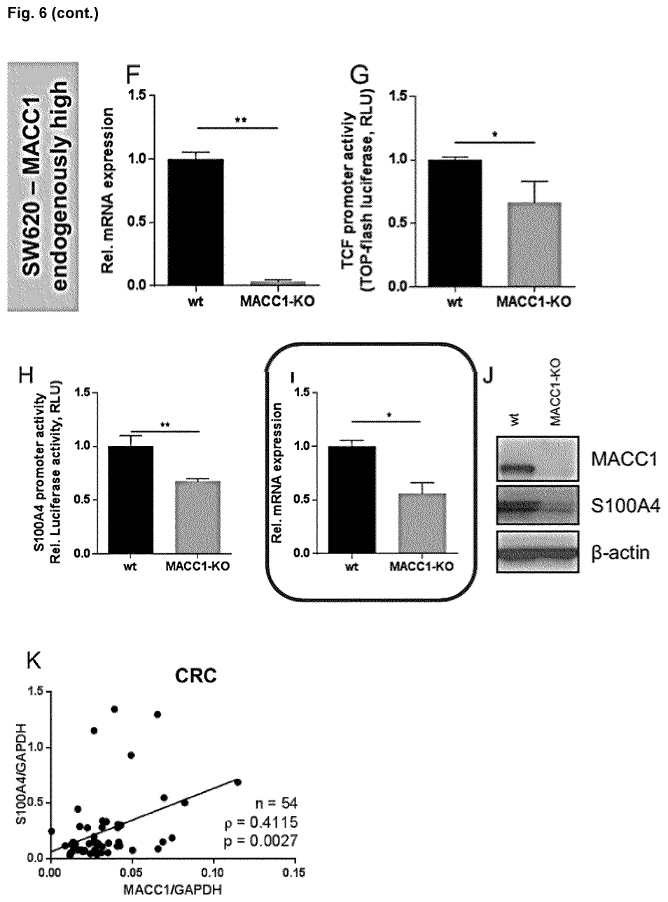

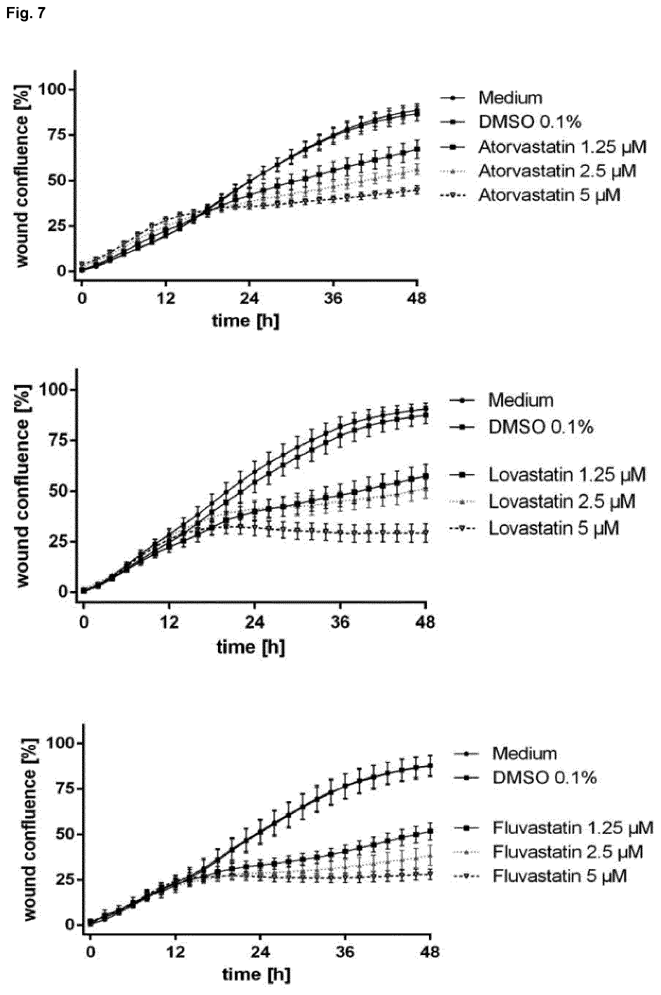

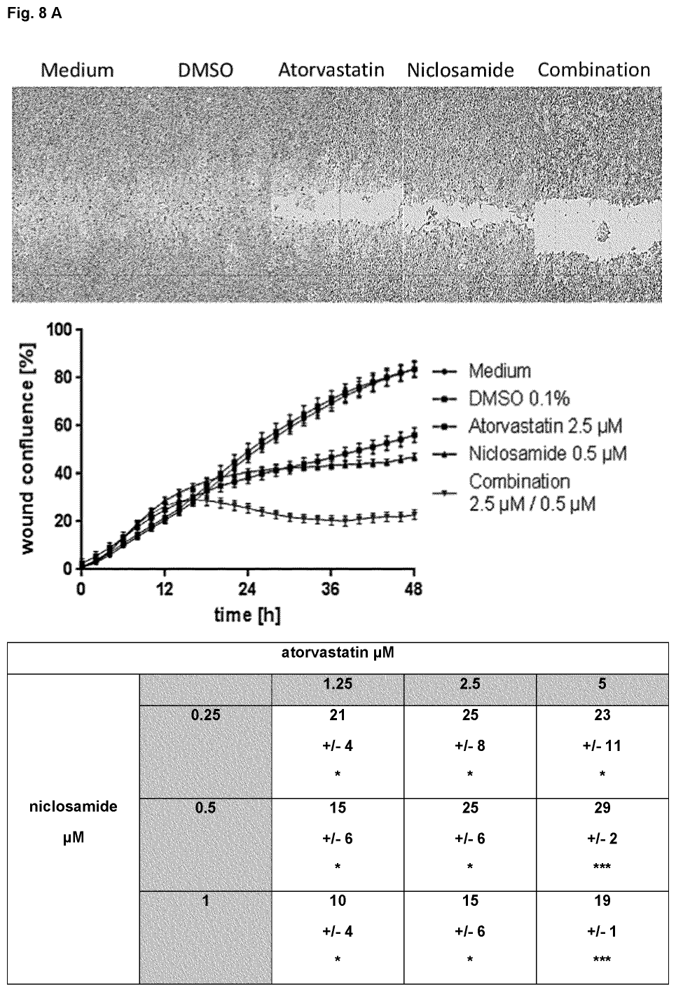

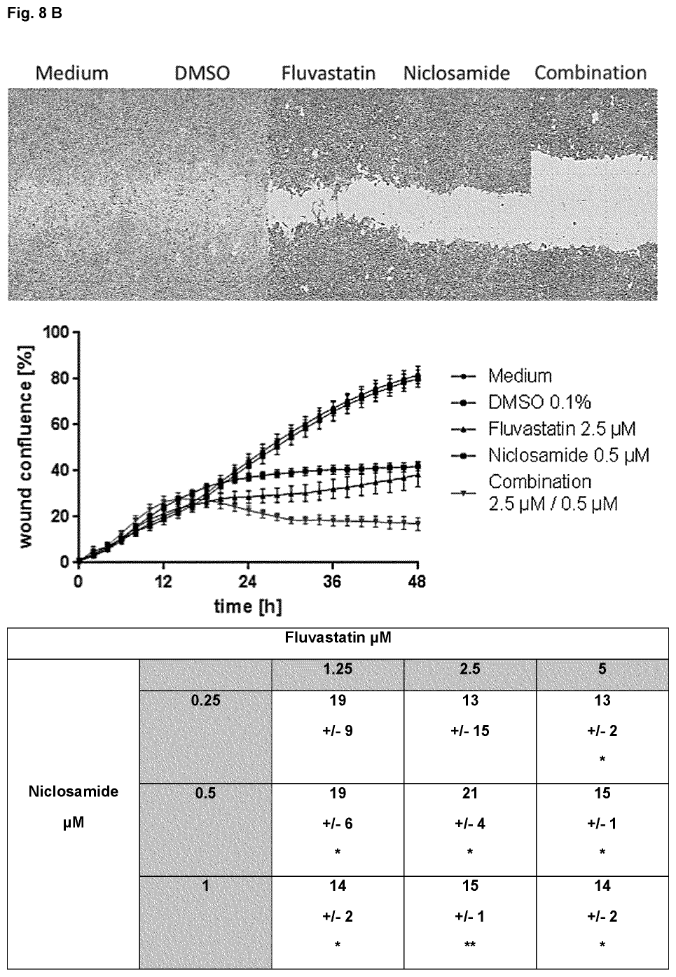

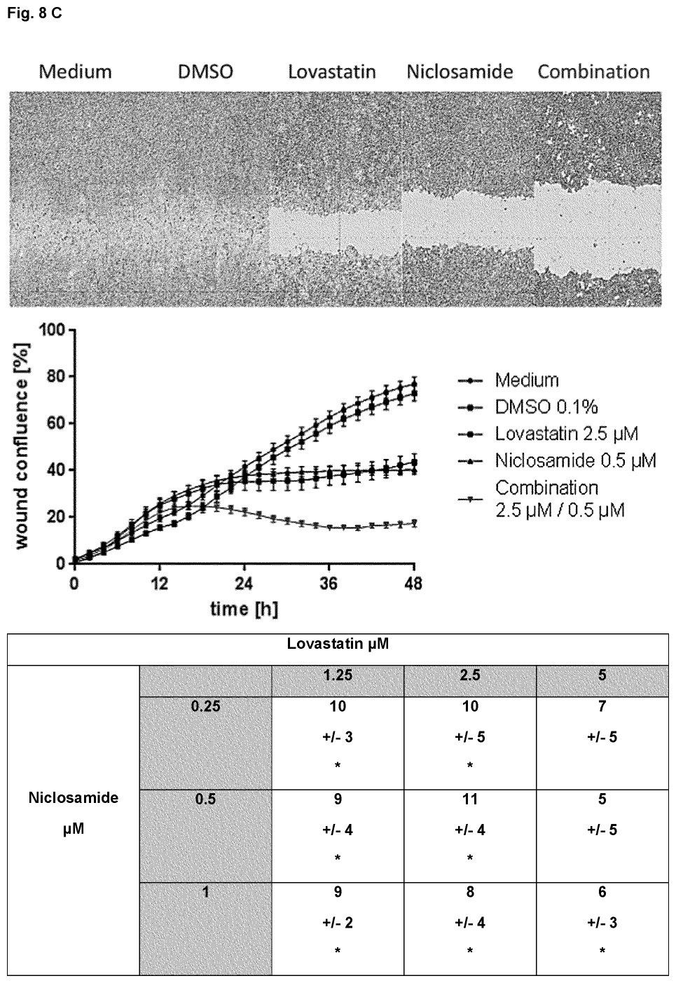

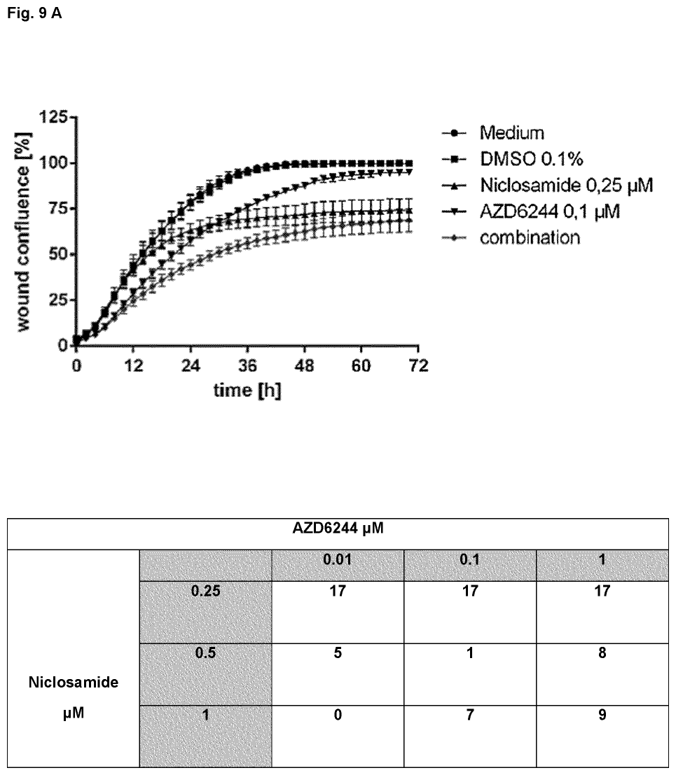

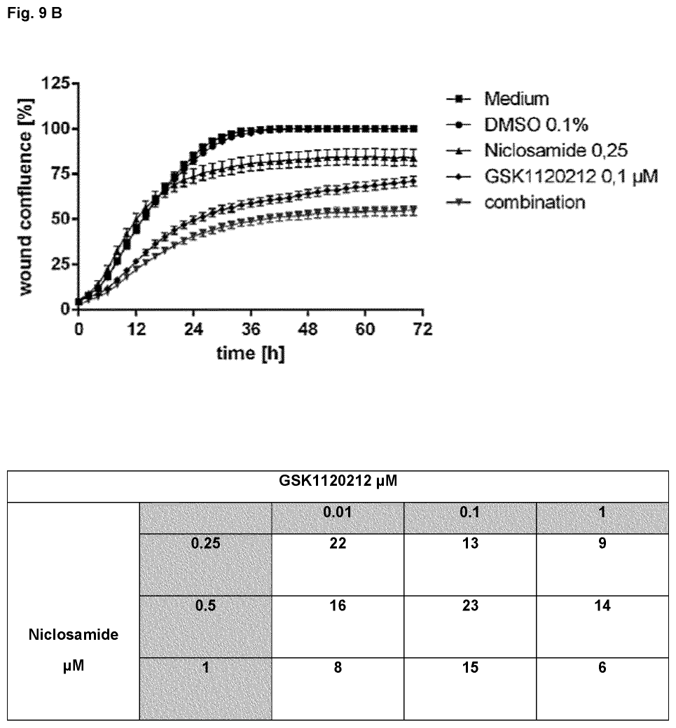

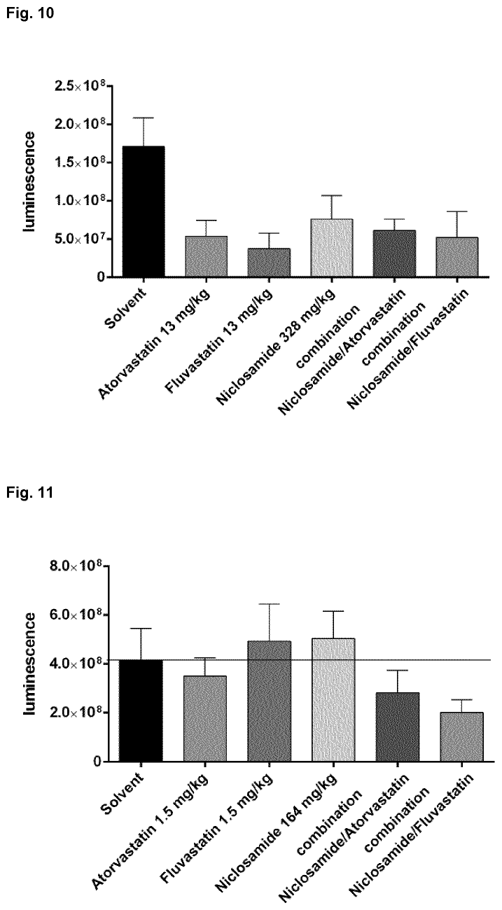

Furthermore, ectopic overexpression of MACC1 induces in vitro the release of factors into the medium, which mediate increased cell motility and invasion in cells of different origins (FIG. 2).

These MACC1 specific supernatants harbor newly secreted S100A4, as determined by SILAC analyses (FIG. 3).

This was further substantiated by MACC1 and S100A4 expression correlation analysis on a publicly available microarray dataset of CRC patients from NCBI GEO database (Tsuji et al. 2012). MACC1 and S100A4 gene expression were positively and significantly correlated regarding metastasis free and overall survival in the CRC patient cohort (p=0.4115, p=0.002, n=54) (FIG. 4).

We identified that MACC1 overexpression increased WNT signaling (TCF promoter) activity and subsequently inducing S100A4 promoter activity in colorectal cancer cells (FIG. 5). This increase in S100A4 promoter activity was further evident in the S100A4 gene and protein expression in these cells.

We also identified that the increase in S100A4 expression in MACC1 ectopically overexpressing cells results from an increased WNT signaling activity in these cells. Conversely, MACC1 knock-down decreased S100A4 promoter activity and gene expression (FIG. 6).

Both MACC1 and S100A4 have been shown to induce migration and metastasis, independently. Our recent work has unraveled a novel mechanism whereby MACC1 induces migration and metastasis via a Wnt/S100A4 axis. Therefore, direct inhibition of Wnt/β-catenin signaling along with MACC1 inhibition represents a viable therapeutic strategy.

In line with this, we tested three different statins in combination with niclosamide. This targets two distinct pathways. All tested statins were able to reduce MACC1 mRNA expression and MACC1 protein (FIG. 1). The expression of MACC1 is inhibited by the statins, thereby MACC1 induced effects are reduced, and niclosamide interferes in Wnt/β-catenin signaling leading to reduced S100A4 expression. First, we confirmed that the all the compounds are able to reduce cellular motility when applied as monotherapy in the wound healing assay. We found a dose dependent reduction of wound closure over time. Cells either non- or DMSO treated closed the wound in the monolayer within 48 h. Compared to this, wound closure of cells treated with monotherapy was delayed for all drugs (FIG. 7).

Next, we tested, if the statins can act synergistically when applied together with niclosamide to reduce cellular motility (FIGS. 8A-C). Here we aim to reduce the drug amount needed to inhibit motility. Compared to the single treatments, inhibition of wound closure was increased when two drugs, niclosamide with one of the statin, were combined. Exemplified for a reduced concentration (2.5 μM for a statin and 0.5 μM for niclosamide) compared to our initial description for HCT116 (5 μM for a statin and 1 μM for niclosamide) we found a synergistically reduced ability to close the wound applied to the HCT116 monolayer.

We have shown that MACC1 is phosphorylated by MEK1 leading to induction of the MACC1 mediated effects. Therefore, we tested, if the MEK1 inhibitors GSK1120212 (trametinib, approved for melanoma treatment) and AZD6244 (selumetinib) act synergistically on cellular motility when combined with niclosamide. Similarly, we applied three different concentrations of trametinib, selumetinib and niclosamide to HCT116 cells and monitored wound closure over time. Here a synergistic activity is detectable, but at a lower level compared to the combination of statins with niclosamide (FIG. 9A-B).

To translate our results to a clinical application, drug combinations composed of niclosamide with a statin were tested in vivo. The combinations niclosamide and fluvastatin, and niclosamide and atorvastatin, were assessed in an in vivo model (mice intrasplenically transplanted with HCT116-CMVp-Luc cells) using oral administration, as described in the methods section above and in FIG. 10-11.

In a first set of experiments, the niclosamide, atorvastatin and fluvastatin were administered both individually and in combination. The statins were administered at 13 mg/kg, and niclosamide at 328 mg/kg, which are doses equivalent to the maximum human dose established for these agents (80 mg daily for the statins, 2 g daily for niclosamide).

As can be seen from FIG. 10, metastasis formation (as determined by luminescence measurement) was reduced when atorvastatin, fluvastatin and niclosamide were applied as single drugs, at these high doses. This metastasis inhibition was also evident in the combinatorial treatment.

This experiment was repeated using reduced doses of niclosamide, atorvastatin and fluvastatin. The statins were administered at 1.5 mg/kg, and niclosamide at 164 mg/kg. These doses correspond to 12.5% of the maximum human dose for the statins and 50% of the maximum human dose for niclosamide (9.2 mg daily for the statins, 1 g daily for niclosamide).

As can be seen from FIG. 11, no reduction in metastasis formation as determined by luminescence measurement was observed when atorvastatin, fluvastatin and niclosamide were applied as single drugs. However, a reduction in metastasis was observed in the combinatorial treatments, further supporting that the combination of active agents leads to an unexpected synergistic effect in reducing metastasis.

Experiments using a combination of lovastatin and niclosamide have also been conducted and show similar results.

In summary, the combinations of a statin with niclosamide, and the MEK1 inhibitor GSK1120212 (trametinib) combined with niclosamide, are able to synergistically reduce cellular motility in the in vitro wound healing assay. Additionally, the combination of lovastatin, fluvastatin or atorvastatin, combined with niclosamide, is superior and synergistic to monotherapy in reducing metastasis formation in the xenograft mouse model.

### Conclusion of the Examples

The inventors show that the close association of S100A4 and MACC1 and their overexpression is associated with poor prognosis of affected CRC patients. The inventors also show the link of these underexplored biomarkers to Wnt-signaling. These findings prompted the inventors to exploit these targets by combined intervention of tumor progression and metastasis formation using repositioned small molecule inhibitors such as niclosamide, statins and MEK1 tyrosine kinase inhibitors. This intervention strategy using drug combinations showed an unexpected synergistic efficacy for metastasis inhibition. This is a strong indication, that targeting of these key pathways and molecules causally involved in cancer metastasis can lead to efficient intervention particularly for patients, who are at high risk for metastasis due to S100A4 and MACC1 overexpression. Thus, treatment of those patients stratified for S100A4 and MACC1 overexpression will be beneficial for these patients and holds enormous promise for clinical use.

1. A pharmaceutical combination, comprising
a. an inhibitor of the Wnt/β-catenin signaling pathway, and
b. an inhibitor of MACC1.

2. The pharmaceutical combination according to claim 1, wherein the inhibitor of the Wnt/β-catenin signaling pathway is an inhibitor of S100A4.

3. The pharmaceutical combination according to claim 2, wherein the inhibitor of S100A4 is niclosamide or derivative thereof, sulindac, calcimycin, ICG001, FH535, LF3, or a phenothiazine.

4. The pharmaceutical combination according to claim 1, wherein the inhibitor of MACC1 is a statin.

5. The pharmaceutical combination according to claim 1, wherein the inhibitor of MACC1 is a MEK1 inhibitor.

6. The pharmaceutical combination according to claim 1, comprising
a. niclosamide, and
b. a statin and/or a MEK1 inhibitor.

7. The pharmaceutical combination according to claim 1, wherein the inhibitor of MACC1 is a statin selected from the group consisting of atorvastatin, lovastatin, fluvastatin, pitarvastatin, pravastatin, rosuvastatin and/or simvastatin.

8. The pharmaceutical combination according to claim 1, wherein the inhibitor of MACC1 is a statin selected from atorvastatin, lovastatin, fluvastatin, pitarvastatin, pravastatin, rosuvastatin and/or simvastatin, and the Wnt/β-catenin signaling pathway is niclosamide or derivative thereof.

9. The pharmaceutical combination according to claim 1, wherein the inhibitor of MACC1 is a MEK1 inhibitor selected from the group consisting of AZD6244 (selumetinib), GSK1120212 (trametinib) and cobimetinib.

10. The pharmaceutical combination according to claim 1, wherein the combination is selected from the group consisting of niclosamide and atorvastatin, niclosamide and lovastatin, niclosamide and fluvastatin, niclosamide and AZD6244 (selumetinib), and niclosamide and GSK1120212 (trametinib).

11. The pharmaceutical combination according to claim 1, wherein (a.) the inhibitor of the Wnt/β-catenin signaling pathway and (b.) the inhibitor of MACC1 have relative amounts of 10000:1 to 1:10000 by weight.

12. The pharmaceutical combination according to claim 1, wherein
the inhibitor of the Wnt/β-catenin signaling pathway is in a pharmaceutical composition in admixture with a pharmaceutically acceptable carrier, and the inhibitor of MACC1 is in a separate pharmaceutical composition in admixture with a pharmaceutically acceptable carrier, or
the inhibitor of the Wnt/β-catenin signaling pathway and the inhibitor of MACC1 are present in a kit, in spatial proximity but in separate containers and/or compositions, or
the inhibitor of the Wnt/β-catenin signaling pathway and the inhibitor of MACC1 are combined in a single pharmaceutical composition in admixture with a pharmaceutically acceptable carrier.

13. A method of treating a tumor disease in a subject in need thereof, comprising administering the pharmaceutical combination according to claim 1 to the subject.

14. The method according to claim 13, wherein the tumor disease is a solid tumor, or selected from the group consisting of gastrointestinal, colorectal, gastric, esophageal, pancreatic, hepatocellular, biliary, lung, nasopharyngeal, renal, bladder, ovarian, brain, bone, head and neck, prostate, melanoma and breast cancer.

15. The method according to claim 13 for treating and/or reducing the risk of tumor metastasis.

16. The method according to claim 13, wherein the tumor cells to be treated exhibit increased expression and/or activity of MACC1 and S100A4 compared to a health control.

17. The method according to claim 13, wherein the subject of treatment exhibits stage 0, I, II, III or IV colorectal cancer, and/or wherein the subject of treatment will undergo and/or has undergone surgery to remove a solid tumor.

18-19. (canceled)

20. The pharmaceutical combination according to claim 7, wherein the statin is selected from the group consisting of atorvastatin, lovastatin and fluvastatin.

21. The pharmaceutical combination according to claim 11, wherein (a.) the inhibitor of the Wnt/β-catenin signaling pathway is niclosamide and (b.) the inhibitor of MACC1 is a statin, and (a.) and (b.) have relative amounts of 1000:1 to 1:1.

22. The pharmaceutical combination according to claim 11, wherein (a.) the inhibitor of the Wnt/β-catenin signaling pathway is niclosamide and (b.) the inhibitor of MACC1 is a MEK1 inhibitor, and (a.) and (b.) have relative amounts of 5000:1 to 1:1.

23. The pharmaceutical combination according to claim 11, wherein (a.) the inhibitor of the Wnt/β-catenin signaling pathway is niclosamide and (b.) the MEK1 inhibitor is GSK1120212 (trametinib), and (a.) and (b.) have relative amounts of 2000:1 to 500:1.

24. The pharmaceutical combination according to claim 11, wherein (a.) the inhibitor of the Wnt/β-catenin signaling pathway is niclosamide and (b.) the MEK1 inhibitor is AZD6244 (selumetinib) or cobimetinib, and (a.) and (b.) have relative amounts of 1000:1 to 1:1.


SEQUENCE LISTING (4 sequences)

SEQ ID NO: 1 - DNA, 21 bp, Homo sapiens
ttcttttgattcctccggtga

SEQ ID NO: 2 - DNA, 20 bp, Homo sapiens
actctgatgggcatgtgctg

SEQ ID NO: 3 - DNA, 20 bp, Homo sapiens
tgtgatggtgtccaccttcc

SEQ ID NO: 4 - DNA, 20 bp, Homo sapiens
cctgttgctgtccaagttgc